(19) United States
(12) Patent Application Publication
(10) Pub. No.: US 2026/0124313 A1
(43) Pub. Date: May 7, 2026

(54) SITE-SPECIFIC COVALENT LIGATION OF HUMAN SERUM ALBUMIN

(71) Applicant: The Regents of the University of California, Oakland, CA (US)

(72) Inventors: Kit S. LAM, Oakland, CA (US); Xingjian YU, Oakland, CA (US); Ruiwu LIU, Oakland, CA (US)

(21) Appl. No.: 19/118,170

(22) PCT Filed: Oct. 6, 2023
(86) PCT No.: PCT/US2023/076195

§ 371 Date: Apr. 3, 2025

Related U.S. Application Data
(60) Provisional application No. 63/378,605, filed on Oct. 6, 2022.

(51) Int. Cl. A61K 47/64; A61K 47/54; A61K 47/65

(52) U.S. Cl. CPC A61K 47/643; A61K 47/542; A61K 47/65

(57) ABSTRACT

Provided herein are albumin selective peptides for covalent ligation to human serum albumin, and conjugates thereof.

### CROSS-REFERENCE TO RELATED APPLICATIONS

This application claims priority to U.S. Provisional Application No. 63/378,605, filed Oct. 6, 2022, which is incorporated herein in its entirety for all purposes.

### STATEMENT AS TO RIGHTS TO INVENTIONS MADE UNDER FEDERALLY SPONSORED RESEARCH AND DEVELOPMENT

This invention was made with Government support under Grant Nos. 1R21AI132458-01 awarded by NIH/NIAID and R01CA247685 awarded by NIH/NCI. The Government has certain rights in this invention.

### BACKGROUND

Human serum albumin (HSA) is the most abundant protein in human blood plasma. There have been many successful precedences leveraging the advantages of HSA as a platform for different diagnostic and therapeutic applications. HSA has been successfully used clinically as a non-covalent carrier for insulin (e.g. Levemir), GLP-1 (e.g. Liraglutide), and paclitaxel (e.g. Abraxane). Efforts have been made to use HSA as a covalent carrier for drug delivery. However, none has been approved for clinical use so far. The development of post-translational chemical modifications that can derivatize native HSA site-specifically under mild reactions will allow researchers to engineer HSA-drug conjugates or supramolecular HSA-based nanostructures, with desirable pharmacokinetic (PK)/pharmacodynamic (PD) properties and protein-adduct ratio, for various biomedical applications. The HSA's only free cysteine (Cys34) makes maleimide chemistry a viable approach to site-specifically modify HSA. One of the concerns for the maleimide-based conjugation strategy, however, is that the resulting thiosuccinimide linkage is unstable under reducing conditions through the retro retro-Michael reaction, or thiol exchange, which poses risks in the performance and safety of the HSA conjugates due to possible unexpected payload release. Furthermore, for targeted drug delivery using HSA as the carrier, it would be advantageous to have additional site-specific ligation strategies that are orthogonal to Cys34, such that targeting ligands and one or more payloads can be reliably and site-specifically conjugated to HSA to form a homogenous conjugate.

The ε-amine on the lysine side chain is another popular site for protein conjugation. The cationic nature of lysine residues at physiological pH makes their distribution relatively on the protein surface and more accessible to conjugation reagents. The conventional lysine conjugation strategy is through reaction with electrophiles, such as N-hydroxysuccinimide ester (NHS-ester), isothiocyanate, or activated aromatic esters. Site-specific conjugation to lysine is more challenging as lysines are abundant in the proteome. For example, for HSA there are 48 lysines in total. These highly reactive electrophiles often fail to differentiate particular lysines, and they tend to react with lysines randomly. Using these non-specific bioconjugation techniques to prepare protein conjugates may compromise the properties of proteins by accidentally labeling physiologically important amino acids. For example, domain IIIB and domain I of HSA are known to be essential for their binding to cell surface FcRn, which is responsible for the recycling of HSA. Modification of surface lysines on these two HSA domains will likely lead to a decrease in circulation half-life of HSA. In the case of antibody-drug conjugates (ADC), non-specific bioconjugation techniques involving lysines could bring unexpected cytotoxicity to the protein conjugates, while on the contrary, homogenous ADCs prepared by site-specific approaches have demonstrated improved therapeutic index. Nevertheless, there have been several studies on performing site-specific conjugation on lysine residues, and site-specific ligations to HSA utilizing Aza-Michael addition between the ε-amine of lysine residues and Michael acceptors, such as α, β-unsaturated sulfonamides and sulfonyl acrylate have also been reported. The chemoselectivity and site-specificity of these strategies originate from kinetic control or delicately tuned substrates such that lysine with higher nucleophilicity and better accessibility can react preferentially. These generic bioconjugation methods, however, are reactive to a variety of proteins. For in vivo applications, it is demanding to develop HSA-exclusive methods to avoid off-target ligation. Surprisingly, the present invention meets this and other needs.

### BRIEF SUMMARY OF THE INVENTION

In one embodiment, the present invention provides a compound of Formula I:

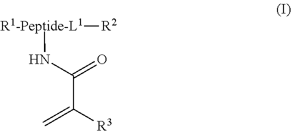

wherein
R¹ is a targeting moiety;
Peptide comprises 3 to 10 amino acids;
L¹ is a linker;
R² is a drug, imaging agent, or affinity ligand; and
R³ is hydrogen or C₁₋₆ alkyl.

In another embodiment, the present invention provides a pharmaceutical composition comprising a compound of the present invention, and a pharmaceutically acceptable excipient.

In another embodiment, the present invention provides a conjugate of Formula IV:

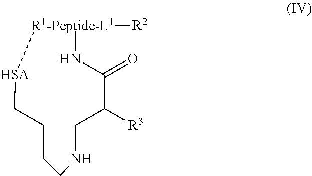

wherein
R¹ is a targeting moiety;
Peptide comprises 3 to 10 amino acids;
L¹ is a linker;
R² is a drug, imaging agent, or affinity ligand;
R³ is hydrogen or C₁₋₆ alkyl; and
HSA is human serum albumin,
wherein the targeting moiety is bound to a first binding site of the HSA.

In another embodiment, the present invention provides a method of preparing a conjugate of the present invention, comprising: forming a reaction mixture comprising a compound of Formula I, II, IIa, or III of the present invention, and human serum albumin, wherein the reaction mixture is at a pH of from 6 to 9, thereby forming the conjugate of Formula IV, IVa, IVb, or IVc of the present invention.

In another embodiment, the present invention provides a method of treating a disease or condition, comprising administering to a subject in need thereof, a therapeutically effective amount of a compound of Formula I, II, IIa, or III of the present invention, forming the conjugate of Formula IV, IVa, IVb, or IVc of the present invention, thereby treating the disease or condition.

### BRIEF DESCRIPTION OF THE DRAWINGS

FIG. 1 shows α, β-unsaturated sulfonamides specifically modified K64 HSA, Sulfonyl acrylate specifically modified K573 of HSA; and site-specific HSA lysine modification via unactivated acrylamide in reactive ligand driven by proximity effect in complex protein mixture such as human serum.

FIG. 2A to 2C shows FIG. 2A Published X-Ray diffraction studies reveal 5 hydrophobic pockets where myristic acid can bind (gray balls). 18 Myristic acid can bind to Domain I and III (red circle) more strongly. FIG. 2B shows design of OBOC libraries. The library compounds were comprised of 3 parts: myristate N-terminus (red), random peptides made by 36 different amino acids (blue), and unactivated acrylamide branched from the side chain of a lysine residue (green). FIG. 2C shows sequence of discovered RAEs (LYL1-4) that are reactive towards HSA.

FIG. 3A to 3I shows chemical structure and characterization of biotin-tagged RAEs LYL1-4 and non-selective 4-nitrophenyl biotin ester (NBE) as positive control used to chemically biotinylate HSA. FIG. 3A shows B-LYL1. FIG. 3B shows B-LYL2. FIG. 3C shows B-LYL3. FIG. 3D shows B-LYL4. FIG. 3E shows 4-nitrophenyl biotin ester. FIG. 3F shows Western blots to detect HSA biotinylated by peptidomimetic RAEs. The conjugation was performed in PBS buffer (pH=7.2) with 20 μM HSA and 100 μM biotinylation reagents for 1 hour at room temperature. FIG. 3G shows Bio-layer interferometry assay measured the intrinsic binding affinity using non-covalent biotin-LYLs. FIG. 3H shows MALDI-TOF Intact MS for HSA-LYL1BT conjugates prepared at various pH in PBS. The conjugates were prepared by mixing 20 μM HSA and 200 μM B-LYL1 for 16 hours. FIG. 3I shows the intensity of corresponding Western blots is proportional to signal intensity from biotinylated HSA in MS and increases as pH elevates.

FIG. 4A to 4E shows structures and characterization data of selected compounds. FIG. 4A shows chemical structure of Biotin-tagged maleimide (B-Mal). FIG. 4B shows FITC-tagged maleimide (FC-Mal). FIG. 4C shows FITC-tagged LYL1 (FC-LYL1) used for selective HSA conjugation in complicated protein matrix. FIG. 4D and FIG. 4E shows comparison between FC-LYL1 and FC-Mal in modifying proteins in serum. Protein SDS-PAGE gels were exposed at green channel (Exc.=490 nm, Emm.=525 nm), then stained by Coomassie Blue. Electrophoresis showed FC-LYL1 can selectively label albumin content. FIG. 4E shows FC-Mal labels multiple proteins.

FIG. 5A to FIG. 5D shows in FIG. 5A the ability of conjugation to specific lysine allows B-LYL1 to modify HSA concurrently with other conjugation strategies in one pot to afford dual-modified HSA conjugates; FIG. 5B shows Western blots for dual-labeled HSA conjugates. B-LYL1 can biotinylate HSA on lysine in the presence of fluorescein-Mal (F-Mal) that modifies cysteine residues. HSA biotinylation was detected by streptavidin Alexa 647 conjugates at red channel (Exc.=594 nm, Emm.=633 nm), while fluorescein tag was detected at green channel (Exc.=490 nm, Emm.=525 nm). FIG. 5C shows Western blots for dual-labeled HSA conjugates using FC-LYL1 and Sulfo-NHS-Biotin. Dual-conjugation status was examined by similar approaches of b. FIG. 5D shows Flow cytometry of LLP2A-HSA-B-LYL1 (20 μM) complex that binds to Jurkat cells. Biotinylated LLP2A ligands (B-LLP2A, 20 μM) were used as the positive control.

FIG. 6 shows Tri-peptide KX3X4 based peptidomimetic OBOC Library.

FIG. 7 shows Tetra-peptide X2 KX3X4 based peptidomimetic OBOC Library.

FIG. 8 shows Penta-peptide X1X2 KX3X4 based peptidomimetic OBOC Library.

FIG. 9 shows a Schematic Illustration for Enzymatic Approach to Find RAE that Can Crosslink HSA.

FIG. 10 shows a Synthetic flow-chart for Biotinylated HSA Reactive Affinity Elements.

FIG. 11 shows Synthesis of FC-LYL-1 (SEQ ID NO:5).

FIG. 12 shows Synthesis of LLP2A-Mal.

FIG. 13 shows Bio-layered Interferometry for NCB-LYL1-4 and NCB-LYL-G

FIG. 14 shows a Steady State Approximation Fitting for Binding between Non-covalent RAE with HSA.

FIG. 15 shows a Schematic Demonstration for Neutravidin Pull-Assay.

FIG. 16 shows aBCA Assay Calibration Curve.

FIG. 17A and FIG. 17B shows a NeutrAvidin Pull-Down Assay Results for Different Biotinylated RAEs (Up) (FIG. 17A) and Western Blots Intensity (Bottom) for Different RAEs, NBE and Blank (FIG. 17B).

FIG. 18 shows a NeutrAvidin Pull-down Assay Showed Conjugation Yield for B-LYL1-4 is pH Dependent. Alkaline pH Promotes Degree of Conjugation.

FIG. 19 shows a Conjugation Yield for B-LYL1-4 at Different pH.

FIG. 20 shows a NeutrAvidin Pull-down Assay Showed Conjugation Yield for B-LYL1-4 is Higher at Elevated RAEs Concentration

FIG. 21 shows a Conjugation Yield at Different RAEs Concentration.

FIG. 22 shows a NeutrAvidin Pull-down Assay Showed Conjugation Yield for B-LYL1-4 is Higher at Prolonged Conjugation Time.

FIG. 23 shows a Conjugation Yield at Different Conjugation Time.

FIG. 24 shows a HSA Sequence (SEQ ID NO:1).

FIG. 25 shows a B-LYL1 Adduct at K225. Peptides at 927.49 m/z (2779.47+3H+) was fragmented (SEQ ID NO:2).

FIG. 26 shows a B-LYL1 Adduct at K225. Peptides at 1056.59 m/z (3166.77+3H+) was fragmented (SEQ ID NO:3).

FIG. 27 shows a Location of K414 and K225 in HSA-Myristate complex (1BJ5). The sites of ligation are close to the myristate binding sites where myristate (MyrB-E) bind.

FIG. 28 shows a Predicted Titration Curve (PDB: 1BJ5) for pK (½) and degree of protonation of LYS-225 (Left) and LYS-414 (Right).

FIG. 29 shows a Electrophoresis and Western Blots for HSA Conjugation in the Presence of IVIG at RAE/HSA Molar Ratio at 1:5 under room temperature for 1 hour Under reducing condition, heavy chains and light chains of IVIG dissociated, resulting in the detection of two bands at 50 kD and 25 kD, respectively, by Western blot (Lane 1). In lane 7, as expected, HSA as well as both heavy and light chains of IVIG and HSA were found to be readily biotinylated by biotin-maleimide conjugate. In contrast, B-LYL1 was able to generate a strong biotin signal for HSA but not for IVIG.

FIG. 30 shows a Conjugation with B-LYL1 and FITC under neutral (pH 7.2) and Alkaline (pH 8.0) in PBS buffer.

FIG. 31 shows a MALDI-TOF Mass Spectrometry for BLYL-1 (M+23, M+39).

FIG. 32 shows a MALDI-TOF Mass Spectrometry for BLYL-2 (Negative Mode).

FIG. 33 shows a MALDI-TOF Mass Spectrometry for BLYL-3 (M+23, M+39).

FIG. 34 shows a MALDI-TOF Mass Spectrometry for BLYL-4 (M+23, M+39).

FIG. 35 shows a MALDI-TOF Mass Spectrometry for BLYL-4.

FIG. 36 shows a MALDI-TOF Mass Spectrometry for Acrylated BLTL1 (Negative Mode).

FIG. 37 shows a MALDI-TOF Mass Spectrometry for NCB-LYL2 (M+23, M+39).

FIG. 38 shows a MALDI-TOF Mass Spectrometry for NCB-LYL3 (M+23, M+39).

FIG. 39 shows a MALDI-TOF Mass Spectrometry for NCB-LYL4 (M+23, M+39).

FIG. 40 shows a MALDI-TOF Mass Spectrometry for NCB-LYLG (M+23, M+39).

FIG. 41 shows a MALDI-TOF Mass Spectrometry for LLP2A-Mal (M+23, M+39).

FIG. 42 shows a MALDI-TOF Mass Spectrometry for B-Mal (M+23, M+39).

FIG. 43 shows a MALDI-TOF Mass Spectrometry for FC-Mal.

FIG. 44 shows a table of compounds of the present invention using iophenoxic acid or ibuprofen as the targeting moiety.

FIG. 45 shows a Western Blot using compounds having iophenoxic acid or ibuprofen as the targeting moiety. The HSA is at a concentration of 1 μM, and the ligand a concentration of 50 μM.

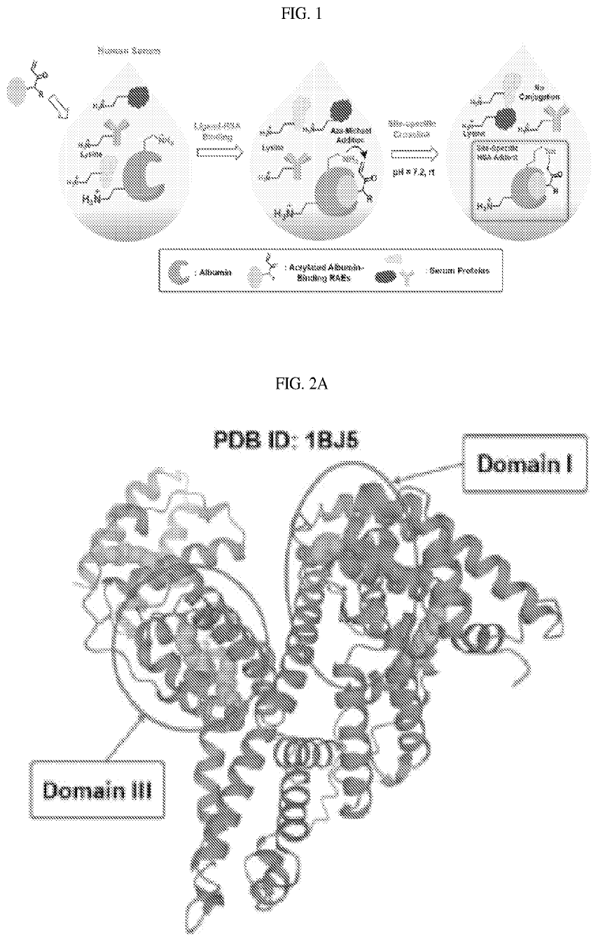

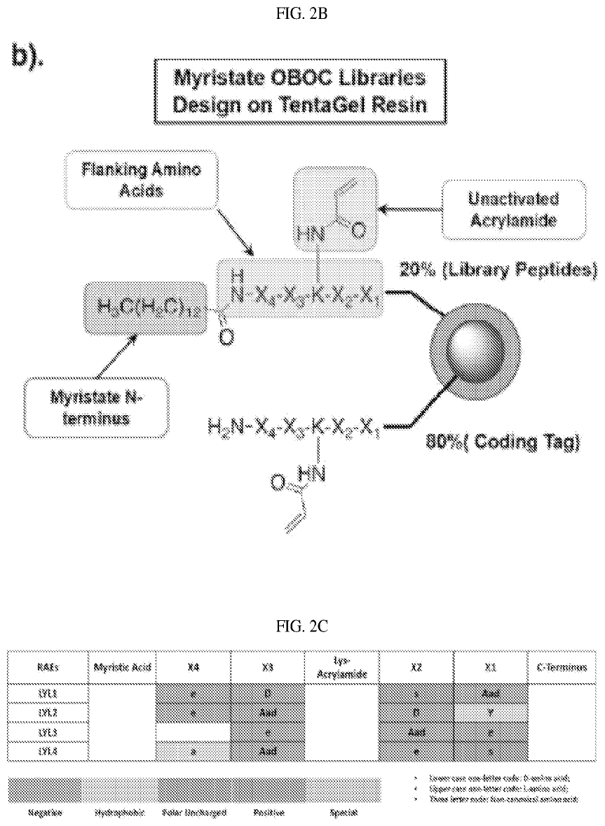

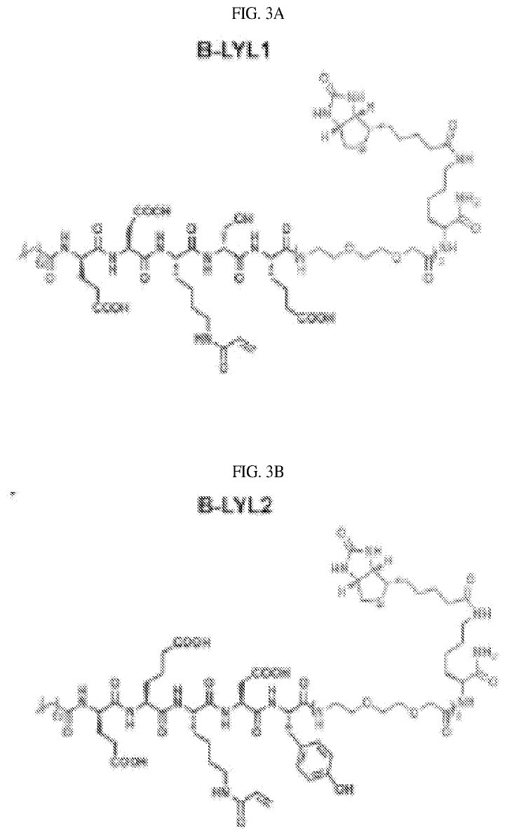

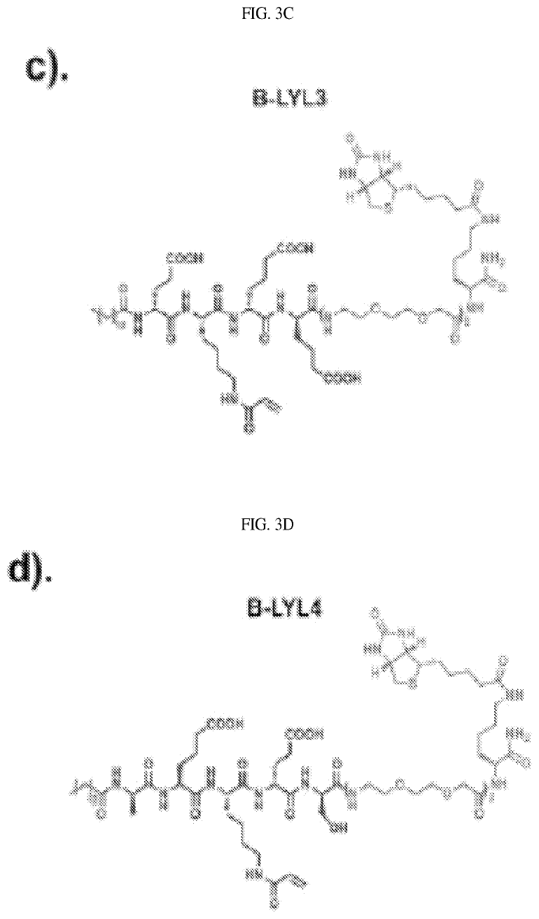

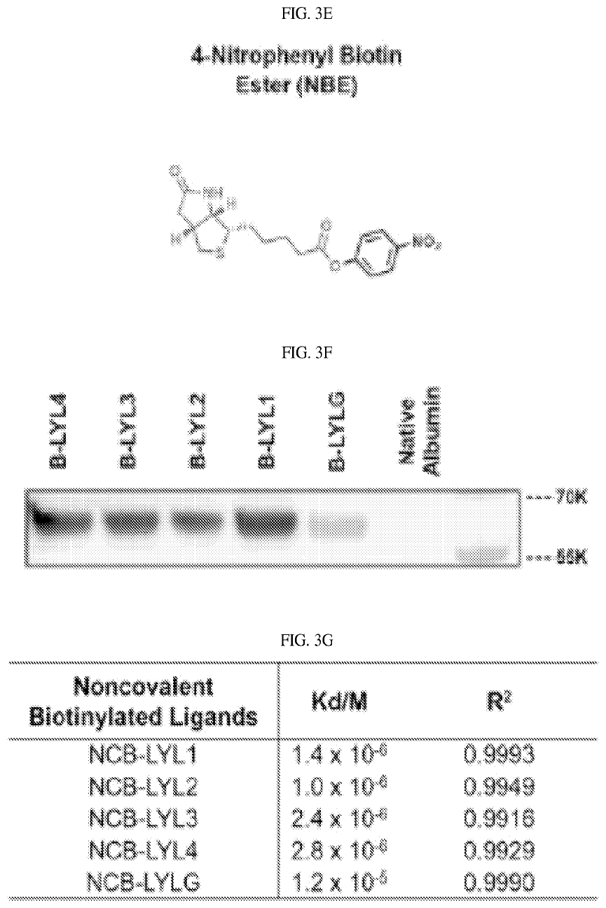

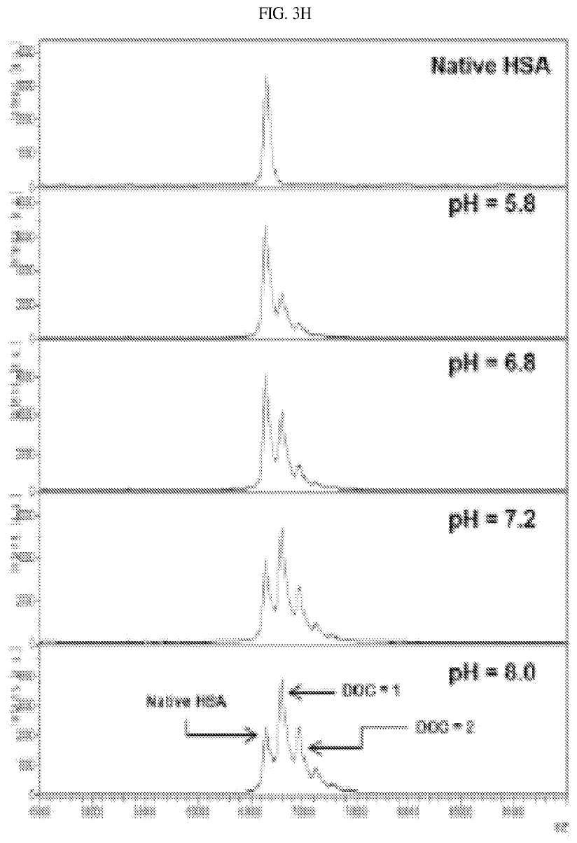

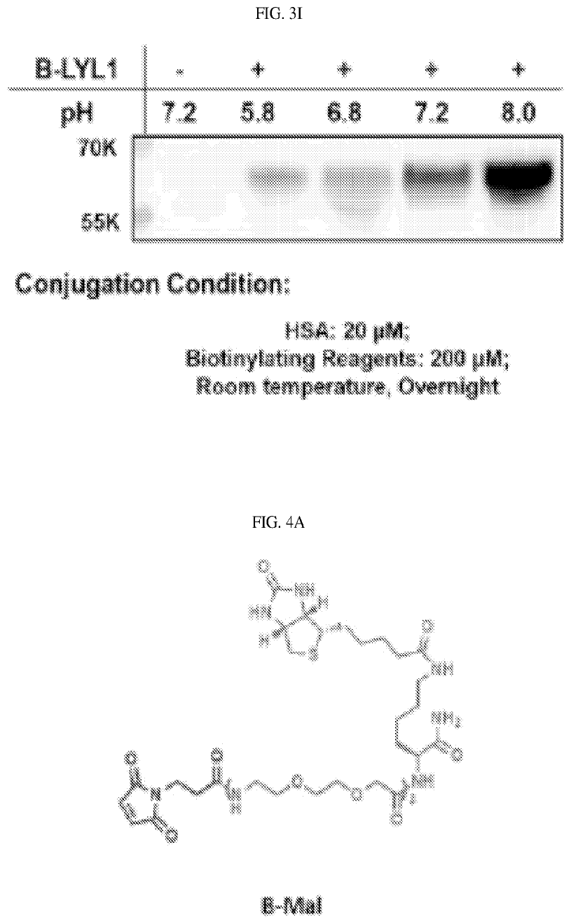

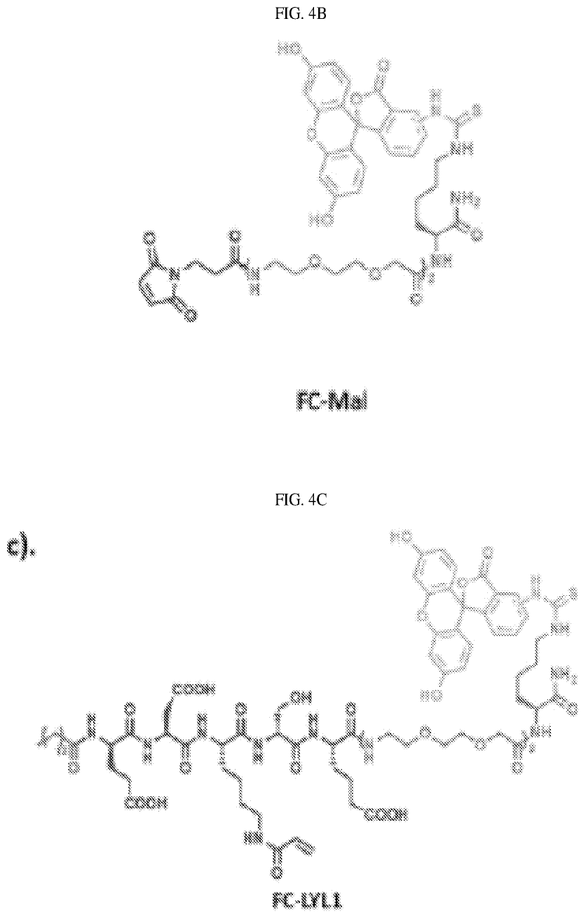

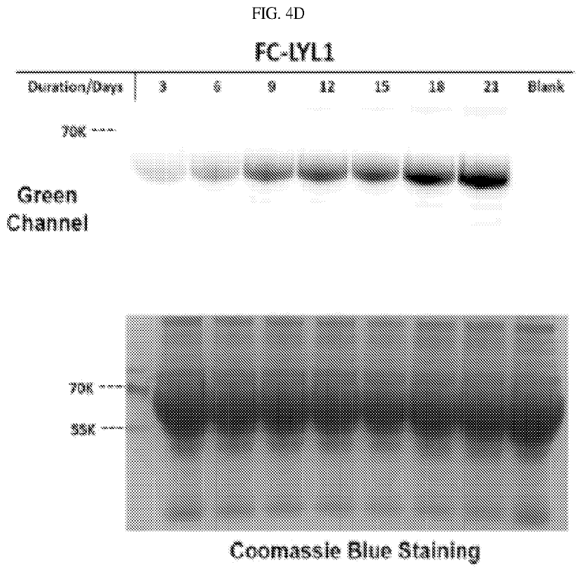

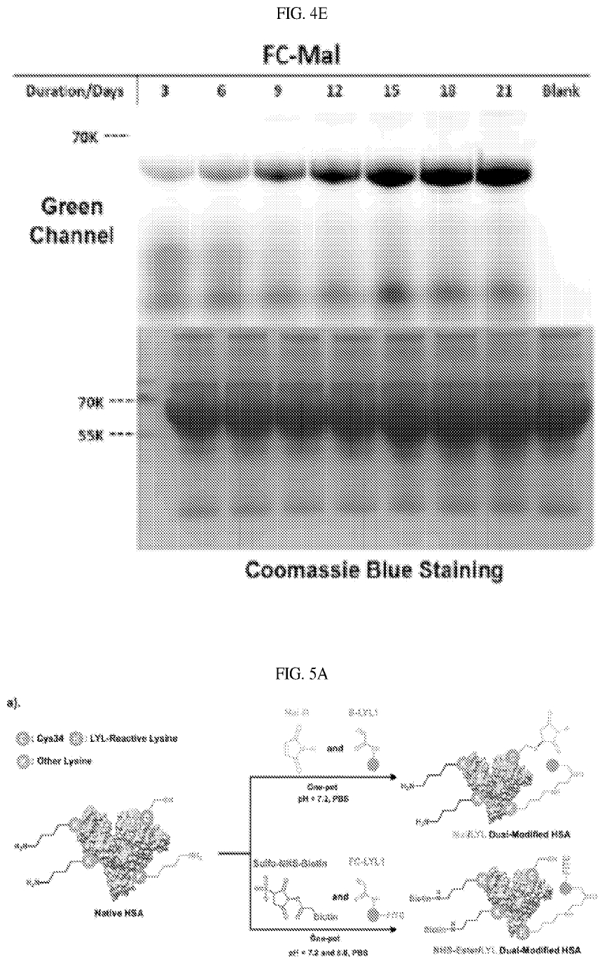

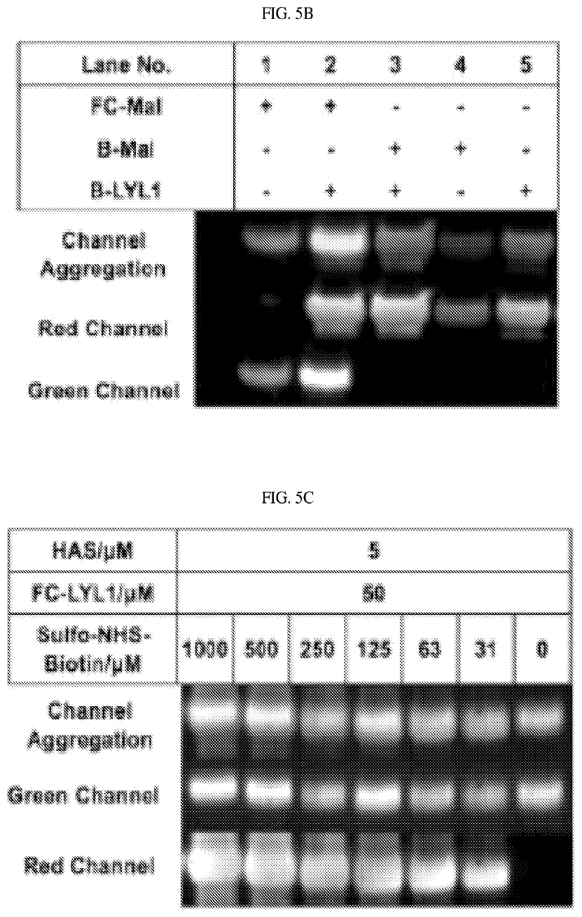

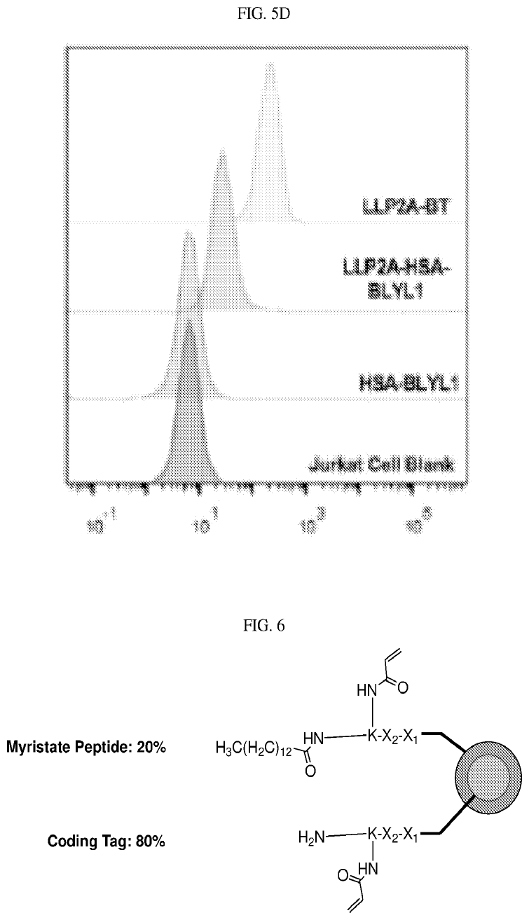

### DETAILED DESCRIPTION OF THE INVENTION

### I. General

The present invention provides acrylamide modified peptides having a targeting moiety that specifically binds to a binding site of a protein such as human serum albumin (HSA). The binding of the targeting moiety to the binding site of HAS increases the nucleophilicity of a proximal amino acid, such as a lysine, which then reacts with the acrylamide to form a covalent linkage of the peptide to the HSA.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Targeting moiety” refers to a peptide or peptidic unit that binds to a binding site a protein, such as human serum albumin (HSA).

“Amino acid” refers to naturally occurring and unnatural amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

| Amino Acid | | | |
|---|---|---|---|
| Name | Symbols | | Systematic name^{c} |
| Alanine | Ala | A | 2-Aminopropanoic acid |
| Arginine | Arg | R | 2-Amino-5-guanidinopentanoic acid |
| Asparagine | Asn | N | 2-Amino-3-carbamoylpropanoic acid |
| Aspartic acid | Asp | D | 2-Aminobutanedioic acid |
| Cysteine | Cys | C | 2-Amino-3-mercaptopropanoic acid |
| Glutamine | Gln | Q | 2-Amino-4-carbamoylbutanoic acid |
| Glutamic acid | Glu | E | 2-Aminopentanedioic acid |
| Glycine | Gly | G | Aminoethanoic acid |
| Histidine | His | H | 2-Amino-3-(1H-imidazol-4-yl)- |
| | | | propanoic acid |
| Isoleucine | Ile | I | 2-Amino-3-methylpentanoic acid^{e} |
| Leucine | Leu | L | 2-Amino-4-methylpentanoic acid |
| Lysine | Lys | K | 2,6-Diaminohexanoic acid |
| Methionine | Met | M | 2-Amino-4-(methylthio)butanoic acid |
| Phenylalanine | Phe | F | 2-Amino-3-phenylpropanoic acid |
| Proline | Pro | P | Pyrrolidine-2-carboxylic acid |
| Serine | Ser | S | 2-Amino-3-hydroxypropanoic acid |
| Threonine | Thr | T | 2-Amino-3-hydroxybutanoic acid^{e} |
| Tryptophan | Trp | W | 2-Amino-3-(1H-indol-3-yl)- |
| | | | propanoic acid |
| Tyrosine | Tyr | Y | 2-Amino-3-(4-hydroxyphenyl)- |
| | | | propanoic acid |
| Valine | Val | V | 2-Amino-3-methylbutanoic acid |

Lower case symbols indicate D-amino acids.

“Amino acid analogs” refers to compounds that have the same basic chemical structure as a naturally occurring amino acid, i.e., an a carbon that is bound to a hydrogen, a carboxyl group, an amino group, and an R group, e.g., homolysine, homoserine, norleucine (Nle), methionine sulfoxide, methionine methyl sulfonium. Such analogs have modified R groups (e.g., norleucine) or modified peptide backbones, but retain the same basic chemical structure as a naturally occurring amino acid.

“Unnatural amino acids” are not encoded by the genetic code and can, but do not necessarily have the same basic structure as a naturally occurring amino acid. Unnatural amino acids include, but are not limited to D-isomers of natural amino acids, L- and D-isomers of following amino acids: azetidinecarboxylic acid, 2-aminoadipic acid (Aad), 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid (Dpr), N-ethylglycine, N-ethylasparagine, homoproline, hydroxylysine, allo-hydroxylysine, 3-hydroxyproline, 4-hydroxyproline (Hyp), isodesmosine, allo-isoleucine, N-methylalanine, N-methylglycine, N-methylisoleucine, N-methylpentylglycine, N-methylvaline, naphthalanine, norvaline (Nva), ornithine, pentylglycine, pipecolic acid, thioproline, 1-aminocyclopropane-1-carboxylic acid (Acpc), citrulline (Cit), homocitrulline (HoCit), D-3-(3-pyridyl)alanine (D-3-Pal), 2-aminoisobutyric acid (Aib), α-aminobutyric acid (Abu), norvaline (Nva), α,γ-diaminobutyric acid (Dbu), D-3-(2-naphthyl)alanine (D-Nal-2), 3-(1-naphthyl)alanine (Nal-1), cyclohexylalanine (Cha), 4-methylphenylalanine [Phe(4-Me)], cyclopropylalanine (Cpa), homoleucine (Hle), D-2-cyclohexylglycine (D-Chg), 3-benzo-thienylalanine (Bta), homoproline (HoPro), β-homoproline (βHoPro), thiazolidine-4-carboxylic acid (Thz), nipecotic acid (Nip), isonipecotic acid (IsoNip), α-aminooctanedioc acid (Asu), diethylglycine (Deg), 4-amino-4-carboxy-1,1-dioxo-tetrahydrothiopyran (Acdt), 1-amino-1-(4-hydroxycyclohexyl) carboxylic acid (Ahch), 1-amino-1-(4-ketocyclohexyl)carboxylic acid (Akch), 4-amino-4-carboxytetrahydropyran (Actp), 3-nitrotyrosine [Tyr(3-NO₂)], 1-amino-1-cyclohexane carboxylic acid (Ach), 2-aminoindane-2-carboxylic acid (Aic), 2-amino-2-naphthylacetic acid (Ana), 4-thiazoylalanine (Tha), allylglycine (Agl), cyclobutylalanine (Cba), D-3-chloro phenylalanine [D-Phe(3-Cl)], D-homophenyalanine [D-HoPhe], 4-benzoyl-L-phenylalanine (Bpa).

“Amino acid mimetics” refers to chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid (i.e., hydrophobic, hydrophilic, positively charged, neutral, negatively charged). Exemplified hydrophobic amino acids include valine, leucine, isoleucine, methionine, phenylalanine, and tryptophan. Exemplified aromatic amino acids include phenylalanine, tyrosine and tryptophan. Exemplified aliphatic amino acids include serine and threonine. Exemplified basic amino acids include lysine, arginine and histidine. Exemplified amino acids with carboxylate side-chains include aspartate and glutamate. Exemplified amino acids with carboxamide side chains include asparagines and glutamine. Conservative substitution tables providing functionally similar amino acids are well known in the art. Such conservatively modified variants are in addition to and do not exclude polymorphic variants, interspecies homologs, and alleles of the invention.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

“Linker” refers to a chemical moiety that links the compound of the present invention to a biological material that targets a specific type of cell, such as a cancer cell, other type of diseased cell, or a normal cell type. Linkers useful in the present invention can be up to 30 atoms or more (including carbon, oxygen, nitrogen sulfur) in length. The types of bonds used to link the linker to the compound and biological molecule of the present invention include, but are not limited to, amides, amines, esters, carbamates, ureas, thioethers, thiocarbamates, thiocarbonate and thioureas. One of skill in the art will appreciate that other types of bonds are useful in the present invention.

“Drug” refers to any compound, small molecule, peptide, protein or antibody that results in the treatment of a disease, disorder, or condition in a subject.

“Imaging agent” refers to a compound capable of being visualized following administration to a subject using a device outside of the subject to determine the location of the imaging agent. Examples of imaging tools include, but are not limited to, positron emission tomography (PET), magnetic resonance imaging (MRI), ultrasound, single photon emission computed tomography (SPECT) and x-ray computed tomography (CT).

“Affinity ligand” refers to a small molecule, peptide, protein, or antibody that has a strong affinity for a biological target such as a specific protein, tissue, cell, or organ of a subject.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, flouromethyl, etc. In some instances, the term “perfluoro” can be used to define a compound or radical where all the hydrogens are replaced with fluorine. For example, perfluoromethyl refers to 1,1,1-trifluoromethyl.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃_s cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

“Cycloalkylene” refers to a cycloalkyl group having the number of carbon atoms indicated, and linking at least two other groups, i.e., a divalent radical. The two moieties linked to the cycloalkylene can be linked to the same atom or different atoms of the cycloalkylene group. Examples of cycloalkylene rings include cyclopropylene, cyclobutylene, cyclopentylene and cyclohexylene, among others. Cycloalkylene groups can be linked 1,1, 1,2, 1,3, or 1,4. The cyclohexylene ring, for example, can adopt a number of conformations, including the boat and chair conformations. The chair conformation of cyclohexylene can have substituents in an axial or equatorial orientation. The divalent nature of the cycloalkylenes results in cis and trans formations where cis refers to both substituents being on the same side (top or bottom) of the cycloalkylene ring, and where trans refers to the substituents being on opposite sides of the cycloalkylene ring. For example, cis-1,2- and cis-1,4-cyclohexylene can have one substituent in the axial orientation and the other substituent in the equatorial orientation, while trans-1,2- and trans-1,4-cyclohexylene have both substituents in the axial or equatorial orientation. cis-1,3-cyclohexylene have both substituents in the axial or equatorial orientation, and trans-1,3-cyclohexylene can have one substituent in the axial orientation and the other substituent in the equatorial orientation. Cycloalkylene groups can be substituted or unsubstituted.

“Heterocycloalkyl” refers to a saturated ring system having from 3 to 12 ring members and from 1 to 4 heteroatoms of N, O and S. Additional heteroatoms can also be useful, including, but not limited to, B, Al, Si and P. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“Heterocyclalkylene” refers to a heterocyclalkyl group, as defined above, linking at least two other groups. The two moieties linked to the heterocyclalkylene can be linked to the same atom or different atoms of the heterocyclalkylene. Heterocycloalkylene groups can be substituted or unsubstituted.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Arylene” refers to an aryl group, as defined above, linking at least two other groups. The two moieties linked to the aryl can be linked to the same atom or different atoms of the aryl. Arylene groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, —S(O)— and —S(O)₂—. Heteroaryl groups can include any number of ring atoms, such as, to 6, 5 to 8, 6 to 8, 5 to 9, 5 to 10, 5 to 11, or 5 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

“Salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

“Binding site” refers to a region of peptide or protein that binds to a targeting moiety with specificity.

“Fatty acid” refers to a carboxylic acid having an aliphatic tail, typically from 4 to carbon atoms long. Fatty acids can be saturated, mono-unsaturated or poly-unsaturated. Fatty acids useful in the present invention also include branched fatty acids such as iso-fatty acids. Examples of fatty acids useful in the present invention, include, but are not limited to, butyric acid (C4), caproic acid (C6), caprylic acid (C8), capric acid (C10), lauric acid (C12), myristic acid (C14), palmitic acid (C16), palmitoleic acid (C16), stearic acid (C18), isostearic acid (C18), oleic acid (C18), vaccenic acid (C18), linoleic acid (C18), alpha-linoleic acid (C18), gamma-linolenic acid (C18), arachidic acid (C20), gadoleic acid (C20), arachidonic acid (C20), eicosapentaenoic acid (C20), behenic acid (C22), erucic acid (C22), docosahexaenoic acid (C22), lignoceric acid (C24) and hexacosanoic acid (C26). One of skill in the art will appreciate that other fatty acids are useful in the present invention.

“Composition” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and deleterious to the recipient thereof.

“Pharmaceutically acceptable excipient” refers to a substance that aids the administration of an active agent to and absorption by a subject. Pharmaceutical excipients useful in the present invention include, but are not limited to, binders, fillers, disintegrants, lubricants, coatings, sweeteners, flavors and colors. One of skill in the art will recognize that other pharmaceutical excipients are useful in the present invention.

“Forming a reaction mixture” refers to the process of bringing into contact at least two distinct species such that they mix together and can react, either modifying one of the initial reactants or forming a third, distinct, species, a product. It should be appreciated, however, the resulting reaction product can be produced directly from a reaction between the added reagents or from an intermediate from one or more of the added reagents which can be produced in the reaction mixture.

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Subject” refers to animals such as mammals, including, but not limited to, primates (e.g., humans), cows, sheep, goats, horses, dogs, cats, rabbits, rats, mice and the like. In certain embodiments, the subject is a human.

“Therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

### III. Compounds

The present invention provides compounds of Formula I capable of specifically binding to a binding site of protein such as human serum albumin (HSA), and forming a covalent bond to the HSA, thereby delivering to the HSA a drug, imaging agent, or affinity ligand.

In some embodiments, the present invention provides a compound of Formula I:

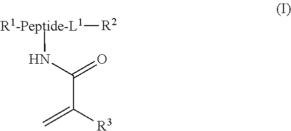

wherein
R¹ is a targeting moiety;
Peptide comprises 3 to 10 amino acids;
L¹ is a linker;
R² is a drug, imaging agent, or affinity ligand; and
R³ is hydrogen or C₁₋₆ alkyl.

In some embodiments, the compound of Formula I is the compound wherein the targeting moiety is specific to a binding site of human serum albumin (HSA). Representative binding sites of HSA include, but are not limited to, Drug Site 1 (Sudlow's site 1 or Sudlow I), Drug site 2 (Sudlow's site 2 or Sudlow II), Drug site 3, DIA, DIB, DIIA, DIIB, DIIIA, DIIIB, and multi-metal binding site. See Frontiers in Immunology, 2015, 5, 682. In some embodiments, the compound of Formula I is the compound wherein the HSA binding site is Sudlow I or Sudlow II. In some embodiments, the compound of Formula I is the compound wherein the targeting moiety is a fatty acid, ibuprofen, or iophenoxic acid.

The acrylamide moiety of Formula I is formed by reaction of an acrylic acid with an amino acid having an amino-substituted side chain such as, but not limited to, lysine, ornithine, homolysine, 2,4-diaminobutanoic acid, and 2,3-diaminopropanoic acid. In some embodiments, the compound of Formula I is the compound wherein at least one amino acid of the Peptide is lysine, ornithine, homolysine, 2,4-diaminobutanoic acid, and 2,3-diaminopropanoic acid. In some embodiments, the compound of Formula I is the compound wherein at least one amino acid of the Peptide is lysine.

In some embodiments, the compound of Formula I is the compound having Formula II:

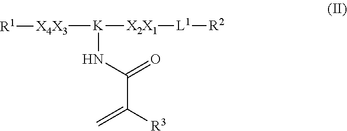

wherein
R¹ is a fatty acid;
X₄ is absent, Aad, D, d, E, e, A, a, V, v, L, I, i, L, l, M, m, F, f, Y, y, W, or w;
X₃ is Aad, D, d, E, or e;
K is lysine;
X₂ is S, s, T, t, N, n, Q, q, Aad, D, d, E, e;
X₁ is Aad, D, d, E, e, A, a, V, v, L, I, i, L, l, M, m, F, f, Y, y, W, w, S, s, T, t, N, n, Q, or q;
L¹ is a linker;
R² is the drug, imaging agent, or affinity ligand; and
R³ is hydrogen or C₁₋₆ alkyl.

In some embodiments, the compound of Formula I or II is the compound wherein the targeting moiety is a fatty acid. The fatty acid can be a saturated fatty acid, a partially unsaturated fatty acid, or a saturated fatty acid. In some embodiments, the compound of Formula I or II is the compound wherein the fatty acid is a saturated fatty acid. In some embodiments, the compound of Formula I or II is the compound wherein the fatty acid is caprylic acid, capric acid, lauric acid, myristic acid, palmitic acid, stearic acid, arachidic acid, behenic acid, lignoceric acid, or cerotic acid. In some embodiments, the compound of Formula I or II is the compound wherein the fatty acid is myristic acid.

In some embodiments, the compound of Formula I or II is the compound wherein at least two of X₄, X₃, X₂ and X₁ are each independently Aad, D, d, E, or e. In some embodiments, the compound of Formula I or II is the compound wherein X₄ is absent, e or a; X₃ is Aad, D, or e; X₂ is s, Aad, D, or e; and X₁ is Aad, Y, e, or s, wherein at least two of X₄, X₃, X₂ and X₁ are each independently Aad, D, or e.

In some embodiments, the compound of Formula I or II is the compound wherein R³ is hydrogen, methyl, ethyl, n-propyl, or isopropyl. In some embodiments, the compound of Formula I or II is the compound wherein R³ is hydrogen or methyl. In some embodiments, the compound of Formula I or II is the compound wherein R³ is hydrogen.

The acrylamide moiety of the compound of Formula I and II can be substituted with electron-donating or electron-withdrawing substituents. For example, the acrylamide can be substituted with hydrogen, C₁₋₆ alkyl, halogen, C₁₋₆ haloalkyl, —OH, —NR^{a}R^{b}, —CN, —NO₂, —C(O)R^{a}, —C(O)NR^{a}R^{b}, cycloalkyl, heterocycloalkyl having 3 to 8 ring members and 1 to 3 heteroatoms each independently N, O, or S, aryl or heteroaryl having 5 to 10 ring members and 1 to 4 heteroatoms each independently N, O, or S, wherein R^{a} and R^{b} are each independently hydrogen or C₁₋₆ alkyl.

In some embodiments, the compound of Formula I or II is the compound having Formula IIa:

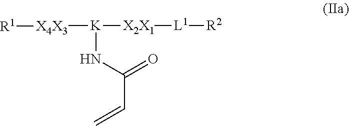

wherein
R¹ is myristic acid;
X₄ is absent, e or a;
X₃ is Aad, D, or e;
K is lysine;
X₂ is s, Aad, D, or e;
X₁ is Aad, Y, e, or s;
L¹ is a linker; and
R² is the drug, imaging agent, or affinity ligand,
wherein at least two of X₄, X₃, X₂ and X₁ are each independently Aad, D, or e.

In some embodiments, the compound of Formula I, II or IIa is the compound having the structure:

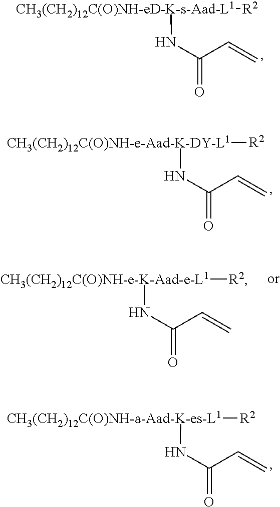

wherein
L¹ is the linker

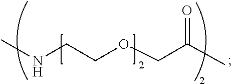

and
R² is the drug, imaging agent, or affinity ligand.

In some embodiments, the compound of Formula I is the compound having Formula III:

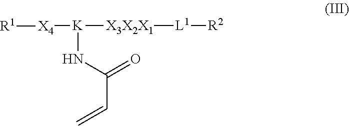

wherein
R¹ is ibuprofen or iophenoxic acid;
X₄ is absent, Acpc, Aib or N;
K is lysine;
X₃ is W, Nle, R, or P;
X₂ is HoCit, R, I, HyP, or Acpc;
X₁ is R, HoPhe, Nle, T, or N;
L¹ is a linker; and
R² is the drug, imaging agent, or affinity ligand,
wherein at least one of X₄, X₃, X₂ and X₁ is Acpc, W, Aib, Nle, HoPhe, or I.

In some embodiments, the compound of Formula I is the compound having Formula III:

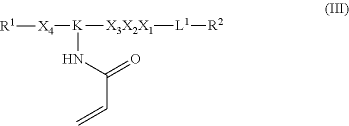

wherein
R¹ is ibuprofen;
X₄ is absent, Acpc, Aib or N;
K is lysine;
X₃ is W, Nle, or R;
X₂ is HoCit, R, or I;
X₁ is R, HoPhe, or Nle;
L¹ is a linker; and
R² is a drug, imaging agent, or affinity ligand,
wherein at least two of X₄, X₃, X₂ and X₁ are each independently Acpc, W, Aib, Nle, HoPhe, or I.

In some embodiments, the compound of Formula I or III is the compound having the structure:

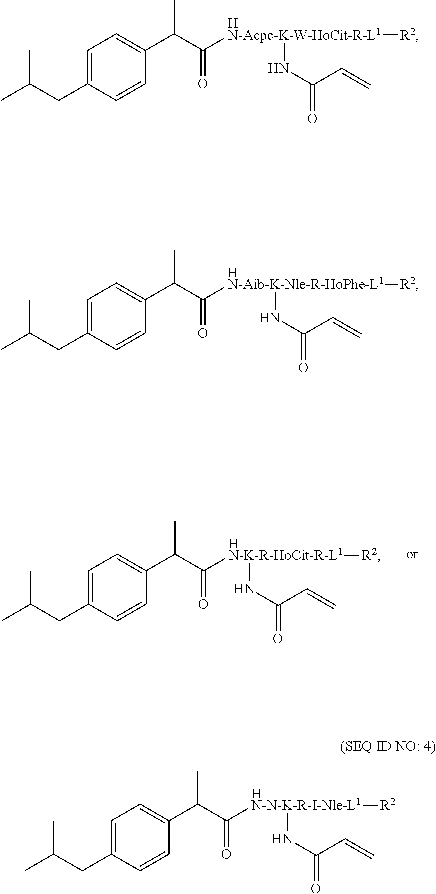

wherein
L¹ is the linker

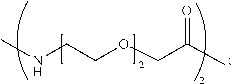

and
R² is the drug, imaging agent, or affinity ligand.

In some embodiments, the compound of Formula I is the compound having Formula III:

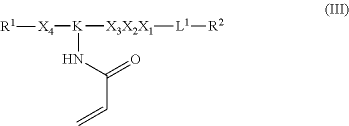

wherein
R¹ is iophenoxic acid;
X₄ is Aib;
K is lysine;
X₃ is R, or P;
X₂ is HyP, or Acpc;
X₁ is T, or N;
L¹ is a linker; and
R² is a drug, imaging agent, or affinity ligand,
wherein at least one of X₄, X₃, X₂ and X₁ is Acpc, or Aib.

In some embodiments, the compound of Formula I or III is the compound having the structure:

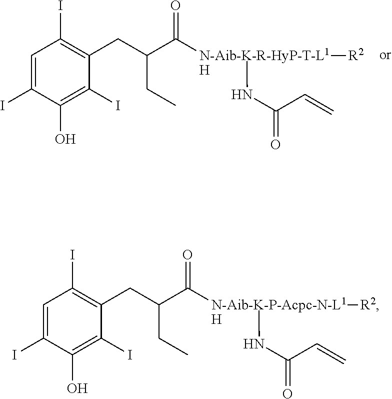

wherein
L¹ is the linker

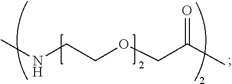

and
R² is the drug, imaging agent, or affinity ligand.

Linkers useful in the present invention include any suitable chemical linkers. In some embodiments, the compound of Formula I, II, IIa, or III is the compound wherein the linker is bis-(2-[2-[2-(amino)ethoxy]ethoxy]acetic acid) (AEEA)2, or EBES6 or polyethylene glycol. In some embodiments, the compound of Formula I, II, IIa, or III is the compound wherein the linker is

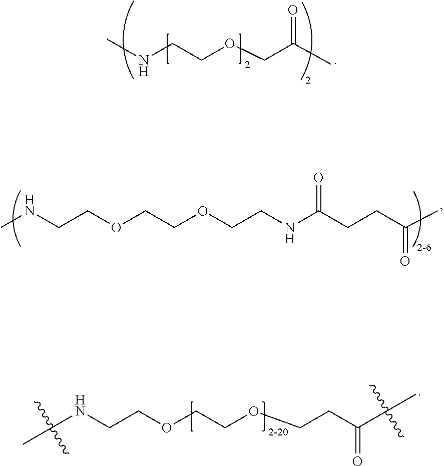

In some embodiments, the compound of Formula I, II, IIa, or III is the compound wherein the linker is

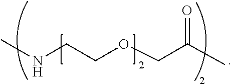

In some embodiments, the compound of Formula I, II, IIa, or III is the compound wherein the linker is EBES6:

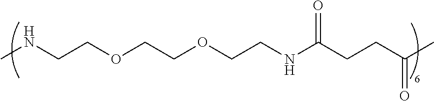

Any suitable drug can be used in the compounds of the present invention. Suitable drugs include, but are not limited to, anti-cancer, antibacterial, antiviral, etc. For example, representative anticancer drugs include, but are not limited to, actinomycin D, azacitidine, bleomycin, bortezomib, dexamethasone, cabazitaxel, carboplatin, cisplatin, daunorubicin, doxorubicin, decitabine, docetacel, epirubicin, etoposide, everolimus, oxaliplatin, idarucicin, imiquimod, riziquimod, irinotecan, lenalidomide, mitomycin C, mitoxantrone, oxaliplatin, paclitaxel, vincristine, vinblastine, streptozocin, temsirolimus, topotecan, or SN-38. Other drugs are useful in the present invention. See also Biochimica et Biophysica Acta 2013, 1830, 5435-5443 for additional drugs.

Any suitable imaging moiety can be used in the compounds of the present invention. The imaging moieties can be imaged by any suitable imaging protocol, such as via fluorescent imaging, radiographic imaging, magnetic resonance imaging, and others. The imaging moiety includes a metal chelator such as, but not limited to, NOTA, DOTA, CB-TE2A, HYNIC, MAG3, DTPA, porphyrin, and a tumor imaging ligand such as, but not limited to, LLP2A for α4β1 integrin of lymphoma and myeloma; LXY30 for α3β1 integrin of solid tumors; LXW64 for αvβ3 integrin of solid tumors; DUPA for PSMA of prostate cancer and vasculature of cancers; and LHRH for the LHRH receptor.

Any suitable affinity ligand can be used in the compounds of the present invention. Affinity ligands useful in the present invention can have an affinity for a specific organ, tissue, or cell of the subject. See Advanced Drug Delivery Reviews 2017, 110-111, 13-37 for tumor affinity ligands useful in the present invention.

### IV. Compositions

In some embodiments, the present invention provides a pharmaceutical composition comprising a compound of Formula I, II, IIa, or III, and a pharmaceutically acceptable excipient.

The compositions of the present invention can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the compounds of the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the compounds of the present invention mixed with a filler or binders such as lactose or starches, lubricants such as talc or magnesium stearate, and, optionally, stabilizers. In soft capsules, the compounds of the present invention may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compounds of the present invention are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compounds of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compounds of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

### V. Administration

The compositions of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound of the present invention can be present in any suitable amount, and can depend on various factors including, but not limited to, weight and age of the subject, state of the disease, etc. Suitable dosage ranges for the compound of the present invention include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds of the present invention can be administered at any suitable frequency, interval and duration. For example, the compound of the present invention can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents, or with adjunctive agents that may not be effective alone, but may contribute to the efficacy of the active agent.

The compounds of the present invention can be co-administered with another active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, i.e., preparing a single pharmaceutical composition including both the compound of the present invention and the active agent. In other embodiments, the compound of the present invention and the active agent can be formulated separately.

The compound of the present invention and the active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the other active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w).

Other dosages and dosage ratios of the compound of the present invention and the active agent are suitable in the compositions and methods of the present invention.

### VI. Conjugates

In some embodiments, the present invention provides a conjugate of Formula IV:

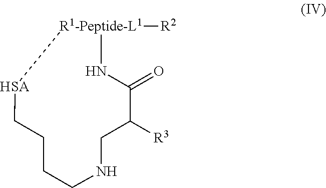

wherein
R¹ is a targeting moiety;
Peptide comprises 3 to 10 amino acids;
L¹ is a linker;
R² is a drug, imaging agent, or affinity ligand;
R³ is hydrogen or C₁₋₆ alkyl; and
HSA is human serum albumin,
wherein the targeting moiety is bound to a first binding site of the HSA.

The targeting moiety is capable of specifically binding to at least one binding site of the protein such as human serum albumin (HSA).

In some embodiments, the conjugate of Formula IV is the conjugate of Formula IVa:

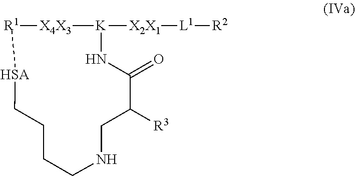

wherein
R¹ is a fatty acid;
X₄ is absent, Aad, D, d, E, e, A, a, V, v, L, I, i, L, l, M, m, F, f, Y, y, W, or w;
X₃ is Aad, D, d, E, or e;
K is lysine;
X₂ is S, s, T, t, N, n, Q, q, Aad, D, d, E, e;
X₁ is Aad, D, d, E, e, A, a, V, v, L, I, i, L, l, M, m, F, f, Y, y, W, w, S, s, T, t, N, n, Q, or q;
L¹ is a linker;
R² is a drug, imaging agent, or affinity ligand;
R³ is hydrogen or C₁₋₆ alkyl; and
HSA is human serum albumin,
wherein the fatty acid is non-covalently bound to the first binding site of the HSA.

In some embodiments, the conjugate of Formula IV or IVa is the conjugate of Formula IVb:

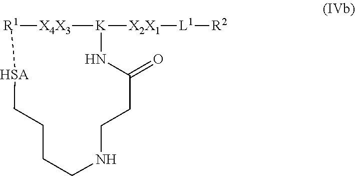

wherein
R¹ is myristic acid;
X₄ is absent, e or a;
X₃ is Aad, D, or e;
K is lysine;
X₂ is s, Aad, D, or e;
X₁ is Aad, Y, e, or s;
L¹ is the linker

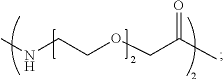

R² is the drug, imaging agent, or affinity ligand; and
HSA is human serum albumin,
wherein the myristic acid is non-covalently bound to the first binding site of the HSA, and
wherein at least two of X₄, X₃, X₂ and X₁ are each independently Aad, D, or e.

In some embodiments, the conjugate of Formula IV is the conjugate of Formula IVc:

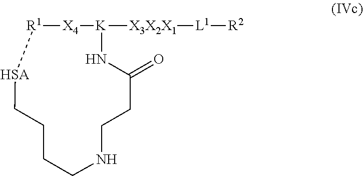

wherein
R¹ is ibuprofen;
X₄ is absent, Acpc, Aib or N;
K is lysine;
X₃ is W, Nle, or R;
X₂ is HoCit, R, or I;
X₁ is R, HoPhe, or Nle;
L¹ is the linker

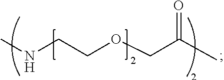

R² is the drug, imaging agent, or affinity ligand; and
HSA is human serum albumin,
wherein the ibuprofen is non-covalently bound to the first binding site of the HSA, and
wherein at least two of X₄, X₃, X₂ and X₁ are each independently Acpc, W, Aib, Nle, HoPhe, or I.

In some embodiments, the conjugate of Formula IV is the conjugate of Formula IVc:

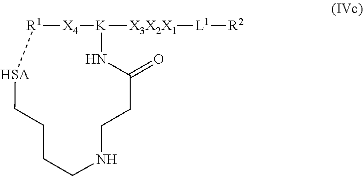

wherein
R¹ is iophenoxic acid;
X₄ is Aib;
K is lysine;
X₃ is R, or P;
X₂ is HyP, or Acpc;
X₁ is T, or N;
L¹ is the linker

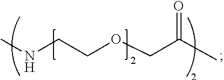

R² is the drug, imaging agent, or affinity ligand; and
HSA is human serum albumin,
wherein the iophenoxic acid is non-covalently bound to the first binding site of the HSA, and
wherein at least one of X₄, X₃, X₂ and X₁ is Acpc, or Aib.

The conjugates of Formula IV, IVa, IVb, and IVc can be prepared by any suitable methods known in the art. In some embodiments, the present invention provides a method of preparing a conjugate of Formula IV, IVa, IVb, or IVc of the present invention, comprising: forming a reaction mixture comprising a compound of Formula I, II, IIa, or III of the present invention, and human serum albumin, wherein the reaction mixture is at a pH of from 6 to 9, thereby forming the conjugate of Formula IV, IVa, IVb, or IVc of the present invention.

The pH of the reaction mixture can be a pH of from 6 to 9, 6 to 8.5, 6 to 8, 6.5 to 8, 6.5 to 7.5, or 7 to 7.5. The pH of the reaction mixture can be 6.0, 6.1, 6.2, 6.3, 6.4, 6.5, 6.6, 6.7, 6.8, 6.9, 7.0, 7.1, 7.2, 7.3, 7.4, or 7.5. In some embodiments, the method of the present invention is the method wherein the reaction mixture is at a pH of from 6.5 to 7.5. In some embodiments, the method of the present invention is the method wherein the reaction mixture is at a pH of about 7.2. In some embodiments, the method of the present invention is the method comprising forming the reaction mixture comprising the compound of Formula IIa, and human serum albumin, wherein the reaction mixture is at a pH of about 7.2, thereby forming the conjugate of Formula IVb. In some embodiments, the method of the present invention is the method comprising forming the reaction mixture comprising the compound of Formula III, and human serum albumin, wherein the reaction mixture is at a pH of about 7.2, thereby forming the conjugate of Formula IVe.

### VII. Methods of Treatment

The compounds of the present invention can be used to treat a variety of diseases. In some embodiments, the present invention provides a method of treating a disease or condition, comprising administering to a subject in need thereof, a therapeutically effective amount of a compound of Formula I, II, IIa, or III of the present invention, forming the conjugate of Formula IV, IVa, IVb, or IVc of the present invention, thereby treating the disease or condition.

### VIII. Examples

The polymeric resins for library/peptide synthesis were acquired through commercial sources. TentaGel S NH₂ resin (90 μm diameter, 0.31 mmol/g NH₂ loading) was purchased from Rapp Polymere (Tubingen, Germany). Rinker amide MBHA resin (0.51 mmol/g NH₂ loading) was purchased from P3 Biosystems (Louisville, KY). All resins were stored at 4° C. before use.

All Fmoc-protected and Boc-protected amino acids in library/peptide synthesis were purchased through commercial sources. The vendors of Fmoc-protected amino acids include Aapptec Inc. (Louisville, KY), Chem-Impex International, Inc. (Wood Dale, IL), and P3 Biosystems (Louisville, KY). All amino acids were stored at 4° C. and used as received without purification.

6-Chloro-1-hydroxy benzotriazole (6-Cl HOBt), 1-[Bis(dimethylamino)methylene]-1H-1,2,3-triazolo[4,5-b]pyridinium 3-oxide hexafluorophosphate (HATU), OxymaPure (Ethyl (hydroxyimino)cyanoacetate) were purchased from Aapptec Inc. (Louisville, KY) and used as received without purification. N, N′-Diisopropylcarbodiimide (DIC), N, N-Diisopropylethylamine (DIEA), trifluoroacetic acid (TFA), Triisopropylsilane (TIS), myristic acid and acetic anhydride were purchased from Sigma Aldrich (St. Louis, MO). Phenylsilane was purchased from TCI America Inc (Portland, ORI. Fluorescein isothiocyanate isomer I was purchased from Chem-Impex International, Inc. (Wood Dale, IL) and stored at −20° C. in the dark before use.

NuPAGE™ 4 to 12% Bis-Tris precast mini-gel, Streptavidin-HRP conjugates, and Streptavidin-Alexa647 conjugated were purchased from ThermoFisher Scientific (Waltham, MA). Anti-HSA antibody alkaline phosphatase conjugate was purchased from Abcam (Cambridge, United Kingdom). Nitrocellulose membrane for western blot transfer, protein standard was purchased from Bio-Rad Laboratories (Hercules, CA). 5-Bromo-4-chloro-3-indolyl phosphate (BCIP) substrate, Human serum albumin (HSA), human serum, bovine serum albumin (BSA), and polysorbate 20 (Tween 20) were purchased from Sigma Aldrich (St. Louis, MO).

All proteins purchased were stored at optimal temperatures based on afflicted specifications. Protein gels were used within the recommended shelf life.

### Example 1: Library Design & Synthesis

The library was composed of 35 natural and unnatural amino acids summarized in Table 1.

TABLE 1
Amino Acid Used in Library
Fmoc- Aib-OH

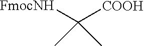

Fmoc-L-Aad(OtBu)-OH

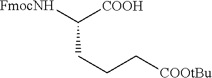

Fmoc-L-Phe(3,4-diCl)-OH

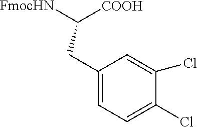

Fmoc-L-Nal-1-OH

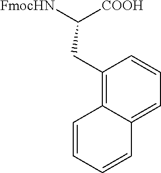

Fmoc-D-HoPhe-OH

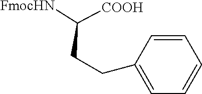

Fmoc-Gly-OH

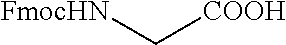

Fmoc-D-Glu(OtBu)-OH

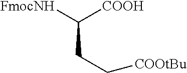

Fmoc-D-Leu-OH

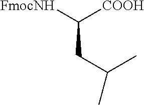

Fmoc-D-Met-OH

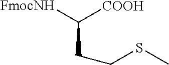

Fmoc-Acpc-OH

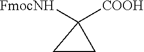

Fmoc-D-3-Pal-OH

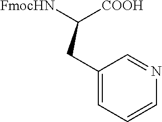

Fmoc-D-Tyr(Me)-OH

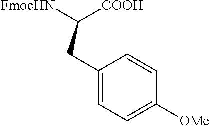

Fmoc-L-Phe(4-Me)-OH

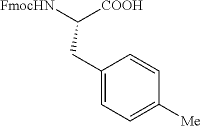

Fmoc-D-Chg-OH

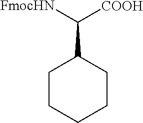

Fmoc-D-Ser(tBu)-OH

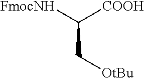

Fmoc-D-Asn(Trt)-OH

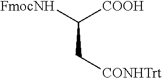

Fmoc-Tyr(tBu)-OH

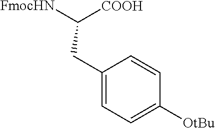

Fmoc-D-Pro-OH

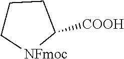

Fmoc-L-HoCit

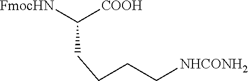

Fmoc-L-Cha-OH

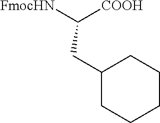

Fmoc-L-Phg-OH

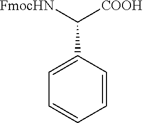

Fmoc-Aic-OH

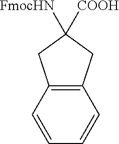

Fmoc-L-Bpa-OH

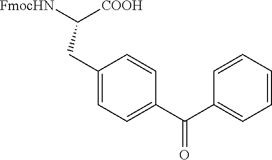

Fmoc-L-Gln(Trt)-OH

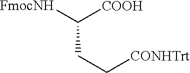

Fmoc-L-Asp(OtBu)-OH

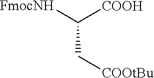

Fmoc-D-Trp(Boc)-OH

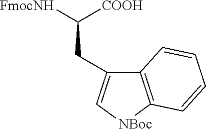

Fmoc-L-Thr(tBu)-OH

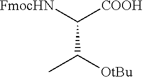

Fmoc-L-Hyp(tBu)-OH

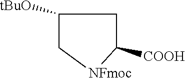

Fmoc-L-Nva-OH

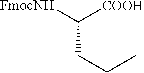

Fmoc-L-Nle-OH

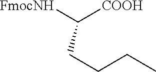

Fmoc-D-Phe(3-Cl)-OH

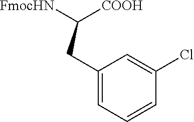

Fmoc-D-Nal-2-OH

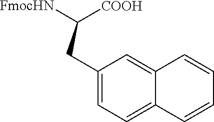

Fmoc-D-Ala-OH

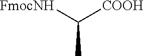

Fmoc-L-Ile-OH

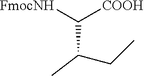

Fmoc-D-Phe-OH

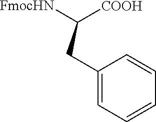

Fmoc-L-Val-OH

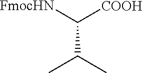

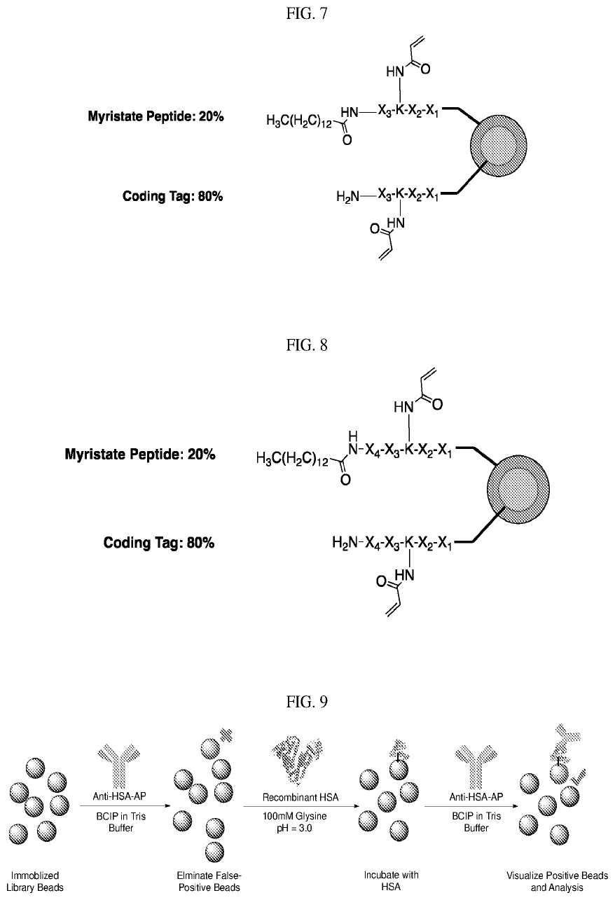

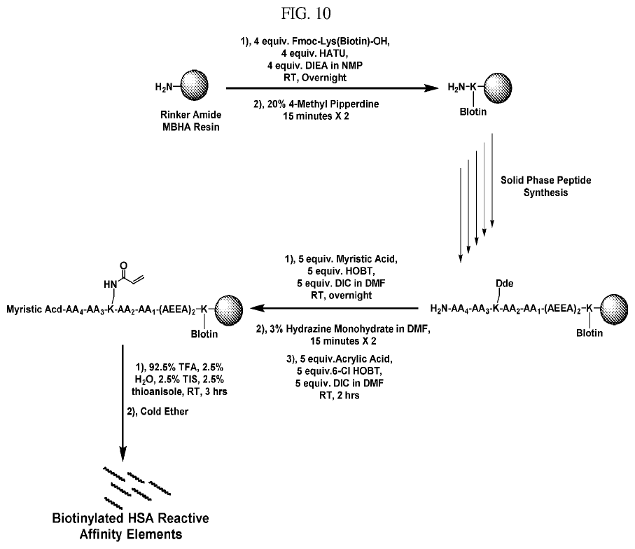

Split-mix strategy was used in library synthesis to yield tri-, tetra- and pentapeptide based peptidomimetic libraries shown in FIGS. 6-8, respectively. 2.0 g of TentaGel beads (NH₂ loading: 0.31 mmol/g) was swollen in DMF overnight before library synthesis. In brief, a library synthesis cycle starts with splitting the beads into 36 5 mL plastic tubes equally. To each vial was added 1 ml of 0.2 M corresponding amino acid solution, 1 ml of 0.2 M 6-Cl HOBt solution, and 50 μL of DIC. The coupling took 2 hours, and the completion of coupling was monitored by the ninhydrin test. After the coupling was done, the beads were combined in a 10 mL disposable polypropylene column with a polyethylene frit and the beads were washed with DMF, methanol and DMF, three times for each, before the addition of 20% 4-methyl piperidine in DMF to remove Fmoc protecting group (5 min, 15 min) and expose the N-terminal amine before next cycle began. Fmoc-Lys(Dde)-OH was coupled at the third cycle using the same coupling method as described above.

Before coupling myristic acid to the N-terminal of the peptides on beads, bi-layer beads were prepared using the bi-phasic solvent approach to achieve 20% binding peptides displayed on the bead outer layer and 80% coding tag reside inside the beads. In brief, the library beads were dried completely and then swollen in water overnight. After water was drained, 178 mg Fmoc-OSu in 40 mL dichloromethane (DCM)/diethyl ether solution (v/v 55:45) was added to the beads, followed by addition of 86 μL DIEA. The column was vigorously shaken for 1 hour. The solution was drained, and the library beads were washed extensively by DMF and DCM. 432 mg Boc-anhydride in DCM was added to protect the N-terminal amino groups of coding peptides. After the Boc protection was done and confirmed by ninhydrin test, 20% 4-methyl piperidine in DMF was added to remove the Fmoc group, followed by coupling with myristic acid using 6-Cl HOBt/DIC. The Dde protecting group on lysine side chain in the library was then removed by 3% hydrazine monohydrate solution in DMF (5 min, 10 min). After extensive washing, 105 mg of acrylic acid (5 equiv.), 262 mg of 6-Cl HOBt (5 equiv.) and 239 μL of DIC (5 equiv.) was added to the beads. The coupling reached completion in 1 hour.

The library beads were washed by DMF, MeOH, (DCM), three times each and dried under vacuum. Global deprotection was achieved with TFA cocktail (v/v, TFA:thioanisole:water:triisopropylsilane=87.5%:5%:5%:2.5%) for 3 hours. Then the TFA cocktail was drained, and the library beads were washed sequentially with DMF, DCM, MeOH, DMF, 50% DMF, water, ethanol, three time each, and stored in 70% ethanol for future screening.

### Enzyme-Linked Immunocolorimetric Screening Procedures

The screening for peptides that covalently target specific domain of HSA is performed in double-incubation approach: incubation with antibody solution to remove the false positive beads before incubation with HSA.

In a separatory column, approximately 100 mg OBOC library (˜300,000 beads) was washed with water for 3 times, followed by PBS-Tween 20-Gelatin buffer (PBSTG, made by 0.05% Tween 20 (v/v), 1% gelatin (w/v), pH=7.4) for 3 times. The library was blocked by PBSTG for 1 hour at room temperature. Then the library was incubated with 2 ml 0.5 μg/ml Anti-HSA-Alkaline Phosphatase conjugate (Anti-HSA-AP) in PBSTG buffer for another 1 hour at room temperature. The antibody solution was drained, and the OBOC library beads were washed by TBS buffer twice. 1.65 mg BCIP substrate was dissolved in 10 ml TBS buffer (pH=8.8), and 1 mL of resulting substrate buffer was added to OBOC library beads. After 1 hour, beads with blue color were picked and discarded, and the rest beads were washed by 8 M guanidine HCl for 3 times, followed by washing with alternating DMF and methanol. Finally, the library beads were washed by PBSTG for 3 times and blocked by PBSTG buffer for another 1 hour. After that the library beads received 1 ml of 7.5 nM HSA solution in PBSTG buffer and the incubation last for 1 hour. Then the beads were washed by PBS for 3 times, followed by 3 times of 100 mM glycine solution (pH=3.0) to elute non-covalently bound HSA. The antibody incubation and color development process were repeated as described above. Beads that bear blue color were picked and sequenced by Edman-degradation micro-sequencer.

### Example 2: Solid Phase Synthesis for RAEs and Derivatives

### Synthesis of Biotinylated Reactive Affinity Elements

Unless specified otherwise, the backbone of myristate peptidomimetics was synthesized by microwave automatic peptide synthesizer (CEM) on Rinker amide MBHA resin (NH₂ loading: 0.51 mmol/g) using 6 equivalents DIC/Oxyma and 6 equivalents of Fmoc-protected amino acids. Each coupling cycle took 2 minutes.

To prepare biotinylated reactive affinity elements and biotinylated non-covalent peptides used in binding affinity study, Fmoc-Lys (Biotin) was first coupled to Rinker amide MBHA resin through 4 equivalents of HATU, Fmoc-Lys(Biotin)-COOH and DIEA in NMP solution at room temperature for overnight. The completion of coupling was monitored by ninhydrin test. Then, two cycles of AEEA linker were coupled to the N-terminal of Lys(Biotin) using 6-Cl HOBt/DIC coupling.

After constructing the backbone of affinity elements, myristate tail was installed on N-terminal of affinity elements by 5 equivalents of myristic acid, 6-Cl HOBt and DIC for overnight at room temperature.

To couple acrylic acid to lysine side chain, after the removal of Dde protecting group by 3% Hydrazine monohydrate, 5 equivalents of acrylic acid, 6-Cl HOBt and DIC were pre-mixed for half an hour before added to the resin. The coupling lasts for 1 hour at room temperature and the completion of coupling was monitored by ninhydrin tests.

To cleave the peptides off the beads, prior to cleavage the beads were washed with DMF, MeOH and DCM and thoroughly dried on vacuum. TFA cocktail (v/v, 90% TFA, 5% thioanisole, 2.5% Water, 2.5% TIS) was prepared and added immediately to the dried beads and the mixture was shaken at a shaking bed for 2 hours. The TFA cocktail solution was then collected and condensed under nitrogen gas. Cold diethyl ether was added to precipitate the peptides and the resulting emulsion was enriched by centrifugation (3000×g, 5 minutes). The ether was then discarded, and the peptides sediments were dissolved by acetonitrile/water mixture (50/50, v/v), followed by purification with Shimadzu LC-20AR Prominence liquid chromatograph suite coupled with a C18 column, starting at the mobile phase composed by ACN/Water=70:30 containing 0.1% (v/v) TFA. The fractions containing desired products were collected or combined. The solvent was removed by lyophilization.

### Synthesis of FC-LYL1

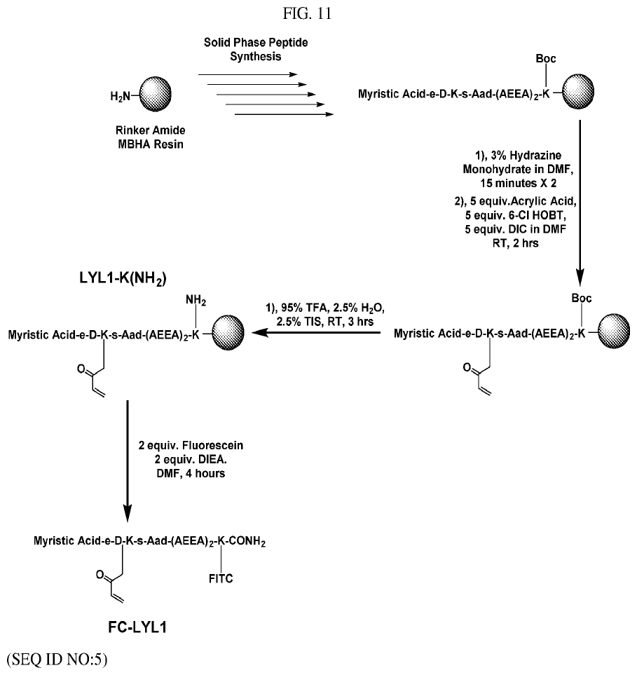

The FC-LYL1 was synthesized from intermediate LYL1-K(NH₂) that is prepared by similar solid phase peptide synthesis (SPPS) strategy described in S4.1. The LYL1-K(NH₂) intermediate was cleaved off the resin by TFA cocktail and purified by reverse-phase HPLC. The purified LYL1-K(NH₂) was crosslinked to FITC through amino-isothiocyanate addition in the presence of 2 equivalents of DIEA (FIG. 11).

### Synthesis of LLP2A-(EBES)₆-Mal

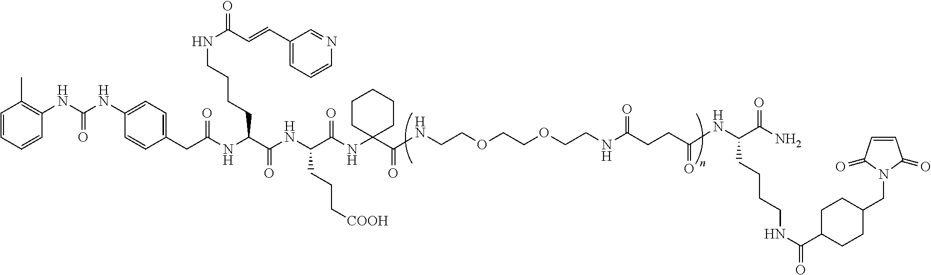

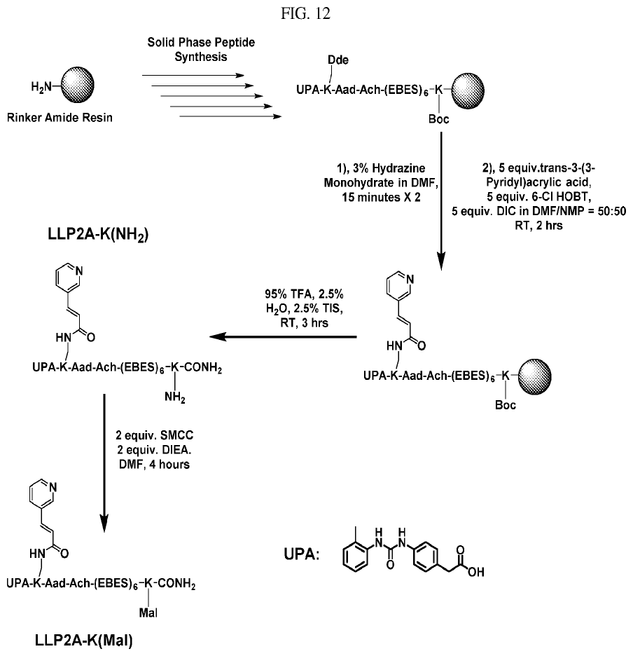

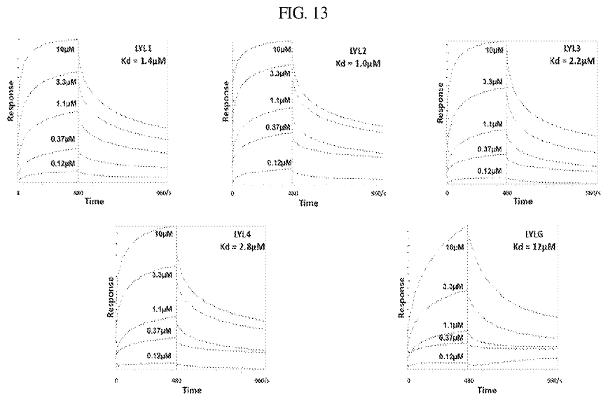

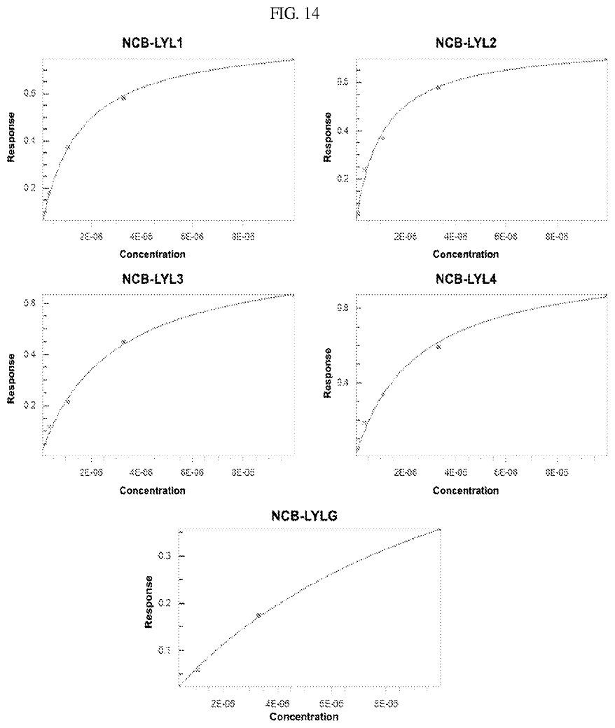

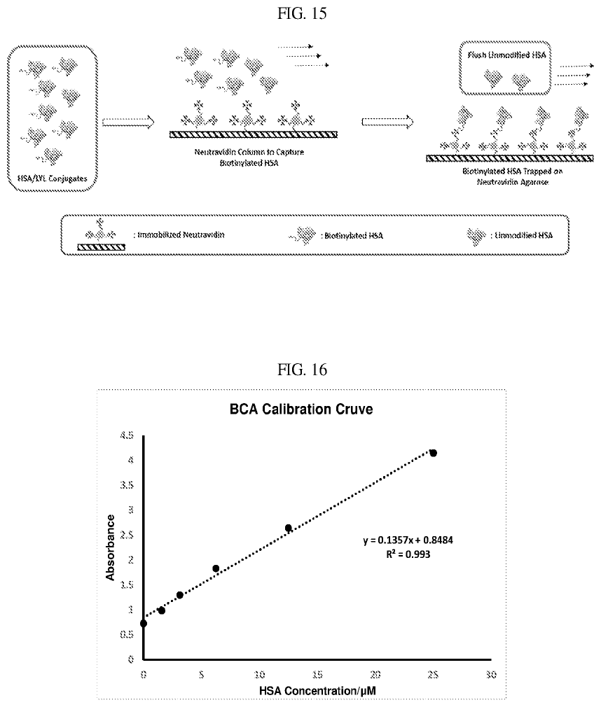

The LLP2A-(EBES)₆-Mal was prepared from the intermediate (LLP2A-(EBES)₆-K(NH₂)) that is synthesized by SPPS strategy as shown in FIG. 12. After purification of intermediate LLP2A-K(NH₂), maleimide was coupled to the amino group of lysine through 2 equivalents of succinimidyl 4-(N-maleimidomethyl) cyclohexane-1-carboxylate (SMCC) in the presence of 2 equivalents of DIEA. The chemical structure is shown in FIG. 13.

### Example 3: BLI Assay for Biotinylated Non-Covalent Peptidomimetics

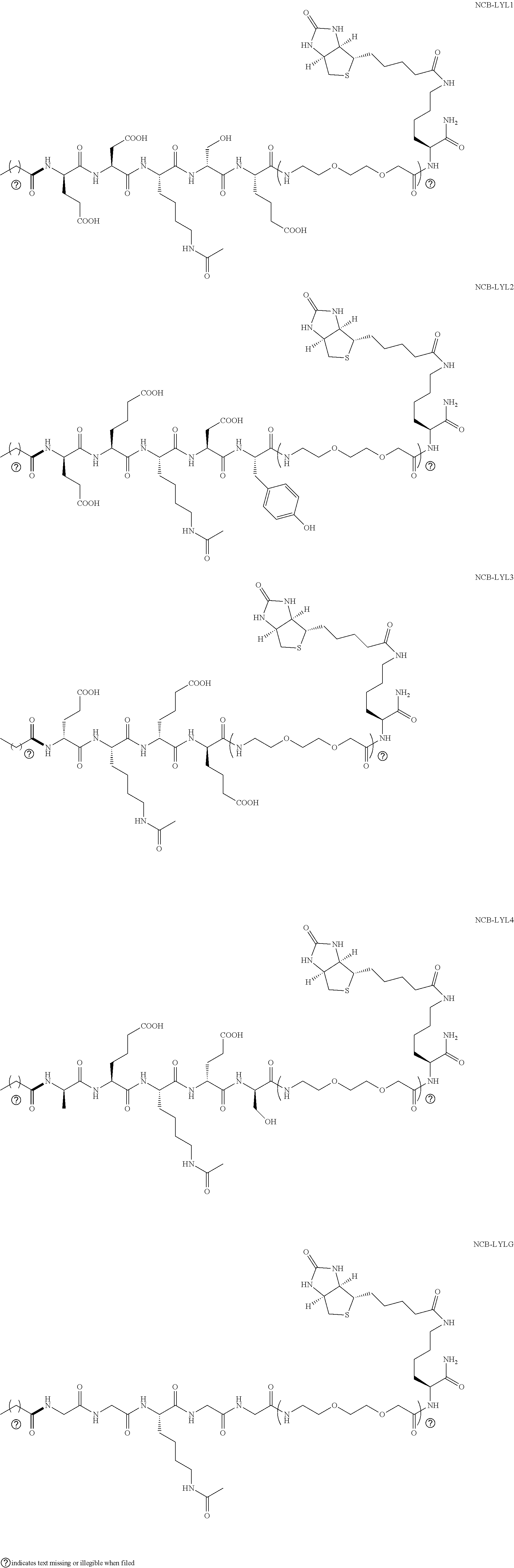

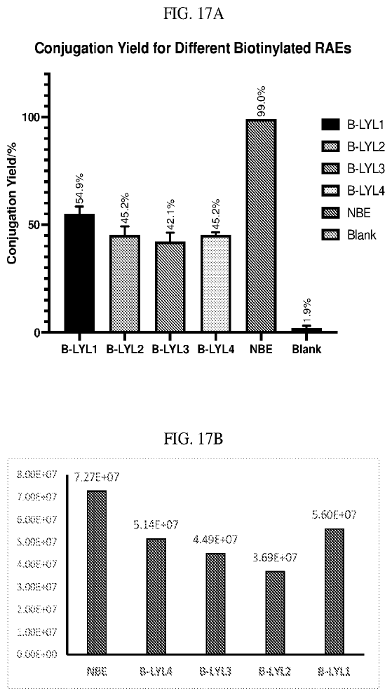

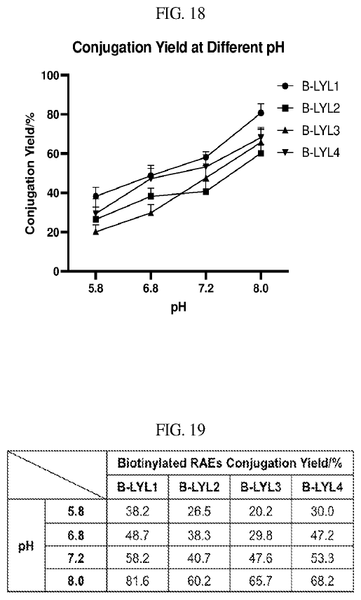

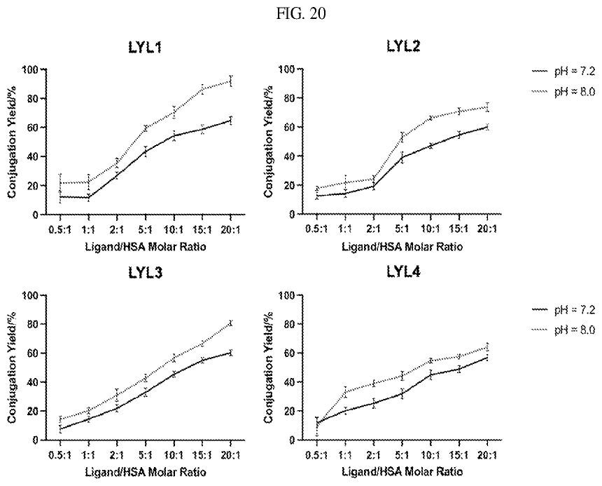

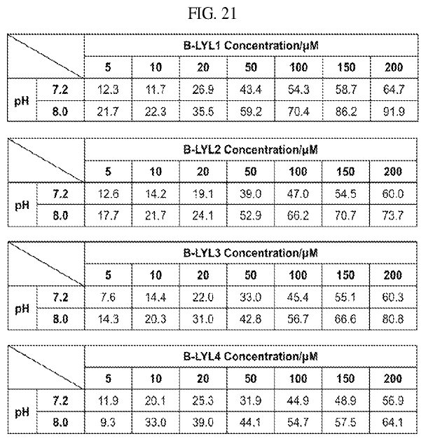

Biotinylated Non-covalent RAEs was synthesized by replacing acrylamide with acetyl group to study the intrinsic binding affinity of peptidomimetics using bio-layer interferometry (BLI) assay. For BLI assay, 1 μM non-covalent biotinylated RAEs (FIG. 14) solution was prepared by kinetic buffer as the bait. As the fish, HSA solution at various concentration prepared by serial dilution using kinetic buffer, and the last well of dilution was filled with kinetic buffer as the reference well. Using 96-well plate, the biosensors were equilibrated in kinetic buffer for 60 s. Then biosensors dipped ligands wells, kinetic buffer wells, HSA well, kinetic buffer wells again for 120 s, 60 s, 480 s, 960 s as loading, baseline, association, and dissociation step, respectively. The data was subtracted by reference wells and processed by ForteBio Data Analysis software to derive the Kd value using steady-state approximation by 1:1 model (FIG. 17-18)

### Example 4: Quantification of Biotinylated HSA Using Neutravidin Pull-Down Assay

### General Procedures to Purify Biotinylated Human Serum Albumin

Zeba spin desalting columns were pre-equilibrated by PBS buffer 3 times on centrifugation (1500×g, 1 minute). After conjugation, HSA-LYL conjugates passed through the equilibrated Zeba desalting column. The flow through was collected and further condensed by a 500 μl ultracentrifugation tube (MW cutoff=3000) at 15000 rpm at 4° C. for 15 minutes. The resulting condensed solution was immediately reconstituted by PBS (pH=7.2), and the condensation process was repeated 2 times to further remove biotinylated peptidomimetics. After that, the condensed HSA conjugate (typically 20-50 μL) was diluted by 100 μL PBS for Neutravidin pull-down assay.

### General Procedure to Determine Conjugation Yield by Neutravidin Pull-Down Assay

Using neutravidin-immobilized agarose, the NeutrAvidin pull-down assay intercepted biotinylated proteins through biotin-avidin interaction. Therefore, the conjugation yield can be determined by measuring the difference caused by NeutrAvidin pull-down.

The yield of conjugation is calculated based on the concentration difference of HSA before and after passing through Neutrvidin agarose, which is calculated by following equation (1):

$$Yield = \frac{{C}_{RSA Stock} - {C}_{RSA Flow - through}}{{C}_{RSA Stock}} \times 100 \% \qquad ( 1 )$$

To perform the pull-down assay, 100 μL slurry of high capacity NeutrAvidin agarose (ThermoFisher Scientific) was loaded to a separatory tube. The resin was equilibrated by binding buffer (PBS buffer, 0.1 M sodium phosphate, 0.15 M sodium chloride, pH=7.2) and washed under centrifugation at low speed (500×g) for 1 minute. The washing was repeated for 3 times. The agarose resin was then dried completely at high speed (5000×g) for 2 minutes to remove water residue. After that, 100 μL purified HSA-LYL conjugate was added, and the resulting mixture was incubated at room temperature for 30 minutes. The flow-through solution was collected by centrifugation and the HSA concentration was determined by Bicinchoninic acid (BCA) assay. 3 replications were performed for pull-down assay for error analysis.

### Bicinchoninic Acid (BCA) Assay

BCA assay was performed using Pierce™ BCA Protein Assay kit to determine the protein concentration in matrix. The BCA working solution was used immediately after preparation. HSA solutions at the concentration of 25.00 μM, 12.50 μM, 6.25 μM, 3.13 μM, 1.56 μM and 0 μM were prepared by binding buffer for calibration curve. The coulometric reactions were performed at 68° C. for 30 minutes and the UV absorbance at 562 nm was measured by plate reader.

### Validation of NeutrAvidin Pull-Down Assay

To 200 μL PBS buffer (0.1 M sodium phosphate, 0.15 M sodium chloride, pH=7.2) was added 1 μL HSA stock solution and 1 μL RAE or 1 μL NBE stock solution to prepare conjugation solution containing 10 μM HSA and 50 μM RAE or 50 μM NBE. The solution was incubated at room temperature for 1 hour. Blank solution was prepared by 10 μM native HSA without modification. The conjugation yield has good relationship with fluorescence intensity of corresponding Western blots. As NeutrAvidin demonstrated very low non-specific binding towards native HSA (<2% uptake), the conjugation yield was calculated without background subtraction.

### Conjugation Yield at Different pH

To 200 μL PBS buffer (0.1 M sodium phosphate, 0.15 M sodium chloride), at different pH was added 1 μL HSA stock solution and 1 μL RAE stock solution to prepare conjugation solution containing 10 μM HSA and 50 μM RAE. The solution was incubated at room temperature for 1 hour.

### Conjugation Yield at Various Concentration

To PBS buffer (0.1 M sodium phosphate, 0.15 M sodium chloride) at pH=7.2 or pH=8.0 was added 1 μL HSA stock solution and various amount of RAE stock solution to prepare conjugation solution containing 10 μM HSA and 5 μM, 10 μM, 20 μM, 50 μM, 100 μM, 150 μM and 200 μM RAEs. Additional PBS buffer was added to make the overall volume of the solution to be 200 μL. The solution was incubated at room temperature for 1 hour.

### Conjugation Yield at for Different Time

To 197 μL PBS buffer at pH=7.2 or pH=8.0 was added 1 μL HSA stock solution and 2 μL RAEs stock solution prepare conjugation solution containing 10 μM HSA and 100 M RAEs. The solution was incubated at room temperature for 1 hour, 2 hours, 3 hours, 4 hours, 5 hours and 24 hours, respectively. The conjugation yield was determined by pull-down assay.

### Example 5: Tryptic Digestion and Proteomics Analysis

### Tryptic Digestion

HSA-BLYL1 conjugate was prepared by 1 mM HSA solution and 10 mM B-LYL1 solutions at alkaline pH (pH=8.0) in room temperature with molar ratio HSA:Biotin-LYL1=1:10 for overnight. The conjugate was purified through desalting column for solvent exchange and condensed by ultracentrifugation before trypsin digestion.

The HSA conjugates were reconstituted in approximately 100 μl of 6.0M urea solution. Then the reducing reagent (DTT stock solution) was added to a final concentration of 5 mM DTT. The mixture was incubated at 37° C. for 30 minutes before the addition of alkylating reagent (Iodoacetamide, IAM) to a final concentration of 15 mM. The alkylation took place at room temperature in dark for 30 minutes, which was later quenched by DTT stock solution. The Lys-C/trypsin was added in a 1:25 (enzyme:protein) ratio and incubate at 37° C. for 4 hours. After that 550 μL 50 mM ammonia bicarbonate (AMBIC) solution was added to dilute the urea and activate trypsin digestion for overnight at 37° C. The conjugates were cleaned-up by MiniSpin™ columns (The Nest Group., Inc) for desating by LC-MS water and acetonitrile.

### Database Searching Parameters

Charge state deconvolution and deisotoping were not performed. All MS/MS samples were analyzed using X!Tandem (The GPM, thegpm.org; version X!Tandem Alanine (2017.2.1.4)). X!Tandem was set up to search the 20190304-human-FRDB-wcrap-wP62 database (unknown version, 147956 entries) assuming the digestion enzyme trypsin. X!Tandem was searched with a fragment ion mass tolerance of 20 PPM and a parent ion tolerance of 20 PPM. Carbamidomethyl of cysteine and selenocysteine was specified in X!Tandem as a fixed modification. Glu->pyro-Glu of the n-terminus, ammonia-loss of the n-terminus, gln>pyro-Glu of the N-terminus, deamidated of asparagine and glutamine, oxidation of methionine and tryptophan, dioxidation of methionine and tryptophan, c+1528 of cysteine and k+1528 of lysine were specified in X!Tandem as variable modifications.

### Example 6: Computational Prediction for Protonation States of HSA

The pK (½) and protonation states of titratable amino acid residues of HSA was calculated on H++ severer based on 3 different crystal structures of native HSA (PDB ID: 1AO6) and HSA-myristate complex (PDB ID: 1BJ5 and 4L8U). The prediction was performed at pH=7.2, with salinity of 0.15 M, internal dielectric constant at 10 and external dielectric constant at 80. The predicted pK int (hypothetical pK of a group assuming that it does not interact with any other titratable group) and pK (½) (mid-point of a titration curve) was summarized in Table 2.

The calculated pK (½) values are compared with previous calculation and experiments results. K199 has lowest pKa value for 1BJ5 and 1AO6, while the pKa of K564 is lowest in 4L8U. The pKa values for K41, K73, K106, K233, K240 and K534 are higher than 12 for all 3 models.

TABLE 2
Predicted pK Int and pK (½) for HSA-Myristate Complex at neutral pH

| | 1BJ5 | | 1AO6* | | 4L8U | | |
|---|---|---|---|---|---|---|---|
| | pK int | pK (½) | pK int | pK (½) | pK int | pK (½) | Remark |
| LYS-12 | 9.92 | 11.19 | 10.08 | 11.91 | 9.97 | 11.08 | |
| LYS-20 | 9.72 | >12.00 | 9.70 | >12.00 | 9.74 | 11.95 | |
| LYS-41 | 10.10 | 11.20 | 9.82 | 11.43 | 10.22 | >12.00 | |
| LYS-51 | 10.06 | 11.74 | 9.66 | 11.12 | 9.67 | >12.00 | |
| LYS-64 | 10.00 | 11.67 | 10.23 | 11.58 | 10.42 | 11.95 | Calculation from |
| | | | | | | | Previous |
| | | | | | | | Literature: pKa = |
| | | | | | | | 11.070 |
| LYS-73 | 9.54 | >12.00 | 9.50 | >12.00 | 9.77 | >12.00 | |
| LYS-93 | 9.89 | 11.08 | 9.60 | 11.00 | 9.80 | 11.09 | |
| LYS-106 | 10.08 | >12.00 | 10.03 | >12.00 | 10.22 | >12.00 | |
| LYS-136 | 8.87 | 11.87 | 9.21 | 10.44 | 9.44 | 9.81 | |
| LYS-137 | 9.65 | 11.95 | 9.64 | 10.61 | 9.91 | 10.99 | |
| LYS-159 | 8.81 | 9.12 | 8.97 | 12.00 | 9.72 | 10.72 | |
| LYS-162 | 9.56 | 11.73 | 9.94 | 11.85 | 9.83 | 11.42 | |
| LYS-174 | 9.65 | 11.21 | 9.52 | 11.08 | 9.99 | 10.97 | |
| LYS-181 | 9.48 | 11.93 | 9.25 | 11.11 | 9.96 | 11.68 | |
| LYS-190 | 9.85 | 9.45 | 9.30 | 6.88 | 9.95 | 10.25 | |
| LYS-195 | 9.94 | >12.00 | 9.90 | 11.97 | 9.93 | >12.00 | |
| LYS-199 | 9.26 | 8.25 | 8.85 | 6.23 | 9.42 | 8.95 | Lowest pKa for |
| | | | | | | | 1BJ5 and 1AO6; |
| | | | | | | | Experimentally |
| | | | | | | | most reactive |
| | | | | | | | lysine for Michael |
| | | | | | | | Addition; |
| LYS-205 | 10.56 | 9.65 | 10.69 | 11.62 | 10.44 | 10.79 | |
| LYS-212 | 9.53 | 11.42 | 9.65 | 10.98 | 9.80 | 11.36 | |
| LYS-225 | 9.64 | >12.00 | 9.62 | 11.57 | 9.72 | >12.00 | Site I |
| LYS-233 | 9.30 | >12.00 | 9.82 | >12.0 | 9.80 | >12.00 | |
| LYS-240 | 10.08 | >12.00 | 9.90 | >12.00 | 9.88 | >12.00 | |
| LYS-262 | 10.15 | 11.94 | 10.04 | >12.00 | 10.19 | >12.00 | |
| LYS-274 | 9.67 | 11.43 | 9.43 | >12.00 | 10.06 | >12.00 | |
| LYS-276 | 10.22 | 11.09 | 9.94 | 11.26 | 10.24 | 11.77 | |
| LYS-281 | 9.79 | >12.00 | 9.95 | 11.10 | 9.93 | >12.00 | |
| LYS-286 | 9.85 | 11.24 | 9.93 | 11.22 | 9.55 | 10.92 | |
| LYS-313 | 9.98 | 11.07 | 10.18 | 11.02 | 10.20 | 11.23 | |
| LYS-317 | 10.05 | 10.90 | 10.11 | 11.48 | 10.23 | 11.12 | |
| LYS-323 | 10.10 | >12.00 | 9.98 | 11.75 | 10.08 | >12.00 | |
| LYS-351 | 9.71 | 10.96 | 9.48 | 11.12 | 9.52 | 9.89 | |
| LYS-359 | 10.38 | 10.96 | 10.18 | 10.83 | 10.34 | 11.11 | |
| LYS-372 | 10.02 | 10.95 | 10.11 | 11.11 | 10.22 | 11.30 | |
| LYS-378 | 10.12 | 11.43 | 10.09 | 11.25 | 10.17 | 11.96 | |
| LYS-389 | 10.23 | 11.16 | 10.11 | 10.82 | 10.18 | 11.64 | |
| LYS-402 | 10.03 | 10.27 | 10.23 | 10.96 | 10.20 | 10.52 | |
| LYS-413 | 10.49 | 10.60 | 10.53 | 10.48 | 11.55 | >12.00 | |
| LYS-414 | 10.06 | 9.81 | 10.24 | 10.38 | 10.93 | 11.59 | Site II |
| LYS-432 | 9.85 | 11.10 | 9.76 | 9.85 | 9.58 | 11.58 | |
| LYS-436 | 10.16 | >12.00 | 10.00 | 10.94 | 9.54 | >12.00 | |
| LYS-439 | 10.47 | 10.82 | 10.45 | 10.93 | 10.61 | 11.40 | |
| LYS-444 | 10.15 | 10.69 | 10.01 | 10.07 | 10.12 | 10.58 | |
| LYS-466 | 10.07 | 10.12 | 10.14 | >12.00 | 10.50 | 10.82 | |
| LYS-475 | 10.20 | 11.42 | 10.15 | 10.69 | 10.20 | 10.81 | |
| LYS-500 | 10.51 | 10.95 | 10.45 | 10.90 | 10.06 | >12.00 | |
| LYS-519 | 9.98 | 10.27 | 9.75 | 9.76 | 9.69 | 9.82 | |
| LYS-524 | 8.70 | 9.79 | 9.75 | 10.37 | 9.91 | 11.42 | |
| LYS-525 | 9.57 | 10.95 | 8.70 | 10.41 | 9.36 | 9.76 | |
| LYS-534 | 10.66 | >12.00 | 10.77 | >12.00 | 10.93 | >12.00 | |
| LYS-536 | 8.90 | 11.09 | 8.36 | 11.13 | 10.32 | 10.82 | |
| LYS-538 | 10.61 | 11.23 | 10.42 | 10.85 | 10.13 | 10.27 | |
| LYS-541 | 9.98 | 10.05 | 10.09 | 10.24 | 8.71 | 7.42 | |
| LYS-545 | 10.27 | 11.19 | 9.73 | 9.81 | 10.32 | >12.00 | |
| LYS-557 | 9.79 | >12.00 | 10.09 | 11.46 | 10.16 | 11.55 | |
| LYS-560 | 10.00 | 10.79 | 10.35 | 11.15 | 10.34 | 11.37 | |
| LYS-564 | 8.94 | 10.57 | 10.05 | 11.12 | 6.02 | 7.63 | Lowest pKa for |
| | | | | | | | 4L8U; |
| LYS-573 | 10.14 | 10.65 | 10.18 | 10.92 | 10.43 | 11.27 | Calculation from |
| | | | | | | | Previous |
| | | | | | | | Literature: pKa = |
| | | | | | | | 10.390 |
| LYS-574 | 10.11 | 11.18 | 10.27 | 10.73 | 10.25 | 11.08 | Calculation from |
| | | | | | | | Previous |
| | | | | | | | Literature: pKa = |
| | | | | | | | 10.890 |

*The calculation of 1AO6 was the arithmetic mean of two chains.

### Example 7: General Procedures to Prepare and Characterize HSA-RAEs Conjugates by Electrophoresis and Western Blots

### Preparation of Stock Solutions for Conjugation

2 mM HSA stock solution was prepared by PBS buffer (0.1 M sodium phosphate, 0.15 M sodium chloride, pH=7.2). 10 mM B-LYL1-4 and B-LYLG stock solutions were prepared in DMSO. 1 mM FC-LYL1 stock solution was prepared with PBS buffer. 10 mM FITC stock solution and FC-LYL1 stock solution was prepared by dissolving in DMSO.

### General Procedures for Electrophoresis & Western Blot

Samples for electrophoresis were prepared by 4×LDS sampling buffer, which was premixed with 20% 2-mercapthanol. The mixture was briefly sedimented by centrifugation and heated for 5 minutes at 95° C. to fully break disulfide bonds and reduce protein. Electrophoresis was done by SDS-NuPage 4-12% gradient Bis-Tris acrylamide gels at constant voltage of 120V for 1 hour. Subsequently, the protein on the gel was transferred to nitrocellulose membrane within 1 hour at 100V in 4° C. by wet transfer. The membrane was then blocked by BSA blocking solution (5% BSA, 0.05% Tween 20 in PBS buffer), followed by incubation of streptavidin-Alexa 647 conjugate for 1 hour at room temperature. The streptavidin-Alexa 647 solution was removed, and the membrane was incubated in PBS buffer in dark for 10 minutes. The fluorescence signal was monitored by gel imager station (Bio-rad) under Alexa 647 channel. Coomassie blue staining was done using Safeblue (Invitrogen) solution for one hour to visualize all protein fragment. The stained gel was soaked in DI water for overnight to de-stain background staining.

Preparation of HSA Conjugates using BLYL1-4 and BLYLG

To 200 μL PBS buffer (0.1M sodium phosphate, 0.15M sodium chloride, pH=7.2) was added 2 μL HSA stock solution and 2 μL RAE stock solution to prepare conjugation solution containing 20 M HSA and 100 μM RAE. The conjugate mixture was incubated at room temperature for 1 hour. 10 μL conjugates were taken for electrophoresis and Western blot without further purification.

### HSA/BLYL-1 Conjugation in the Presence of IVIG

To 193 μL PBS buffer (0.1 M sodium phosphate, 0.15 M sodium chloride, pH=7.2) was added 1 μL HSA stock solution, 1 μL BLYL-1 stock solution and 5 μL 0.13 mM IVIG stock solution to prepare conjugation solution containing 10 μM HSA (0.67 mg/ml) and 3.3 μM IVIG (0.5 mg/ml) and 50 μM BLYL-1. Blank and comparative experiments used PBS to replace the portion of RAEs. The ratio of HSA and IVIG concentration resembles the composition of HSA and globulin in human serum. (Assuming serum albumin content in serum: 35 mg/ml, globulin content: 28 mg/ml). The solution was incubated at room temperature for 1 hour. 10 μL conjugates were aliquoted for electrophoresis and electro-blotting without further purification (FIG. 26).

### BLYL-1 Conjugation to Serum-Borne HSA

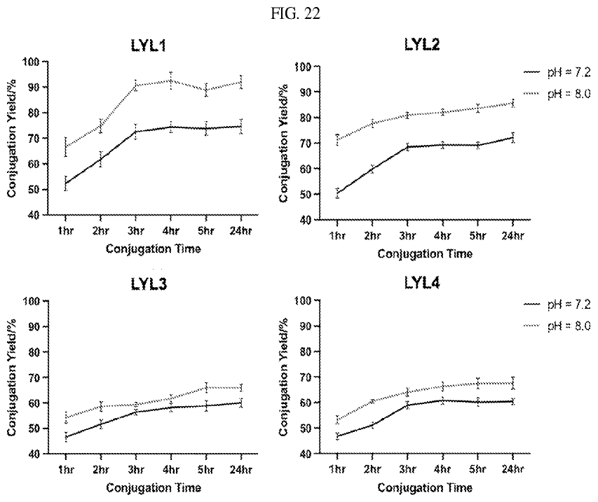

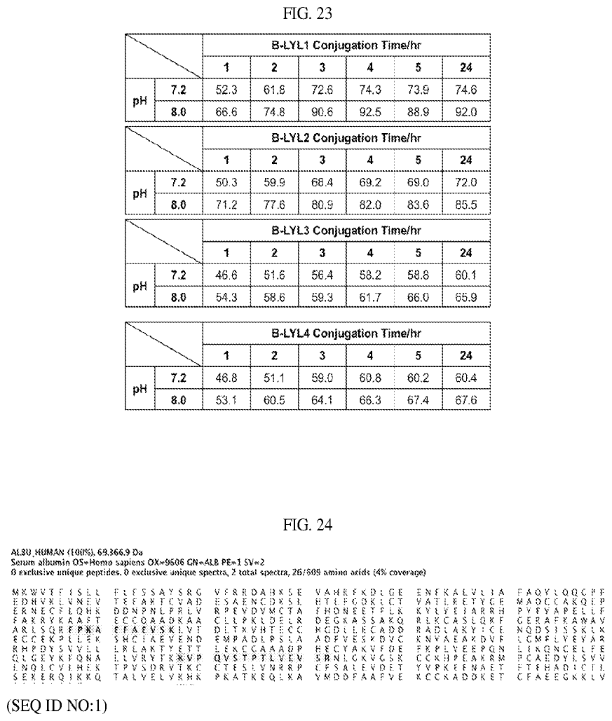

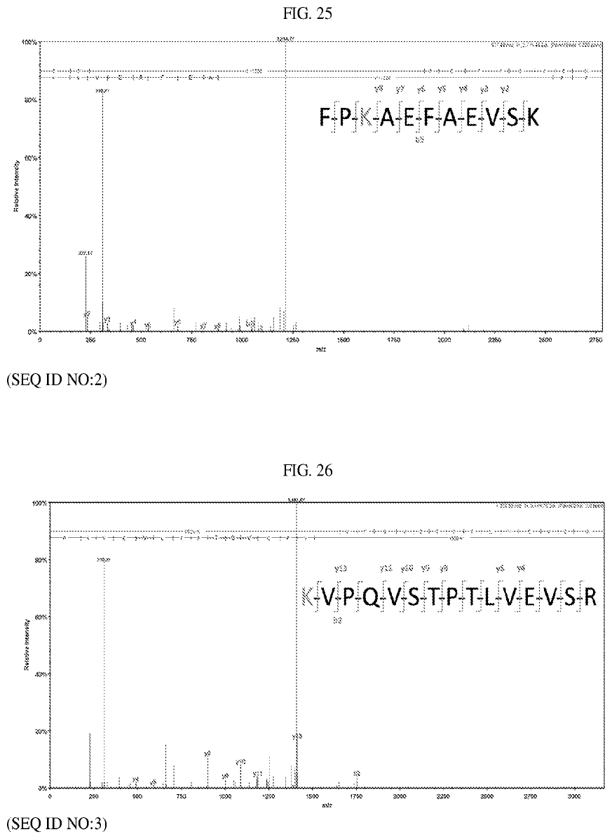

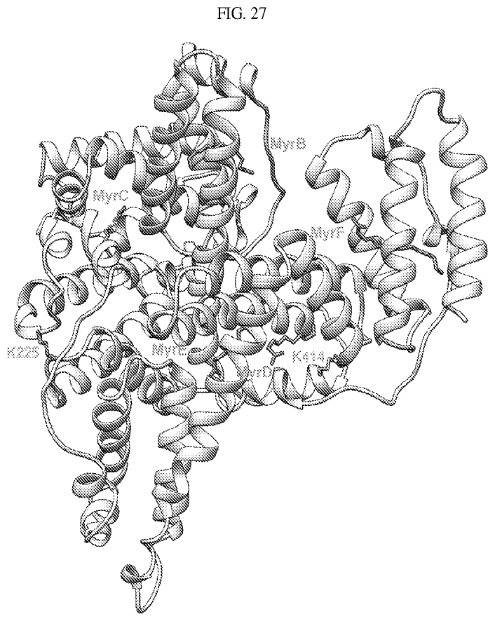

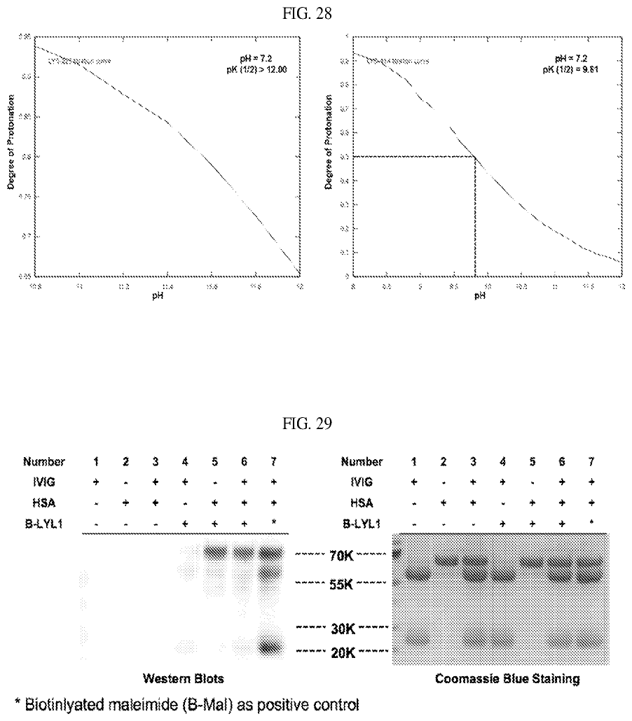

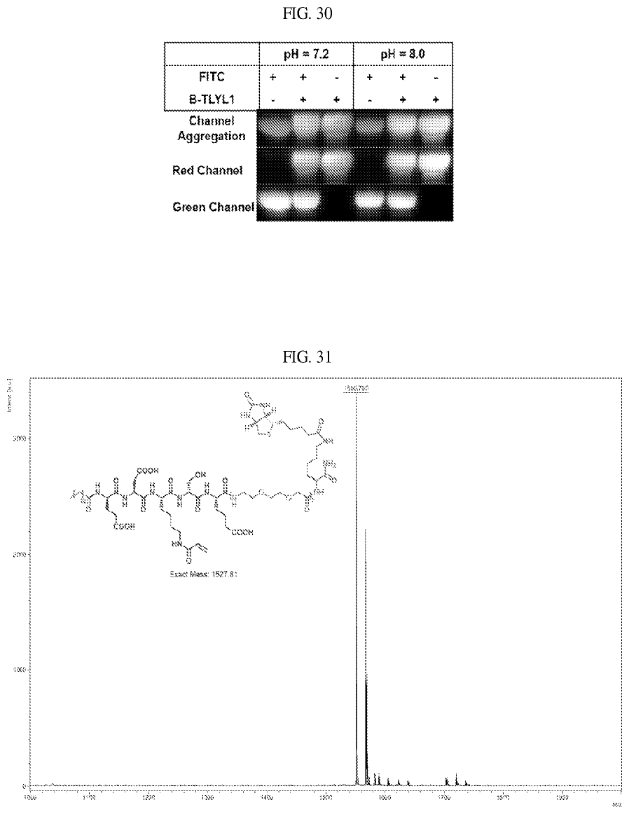

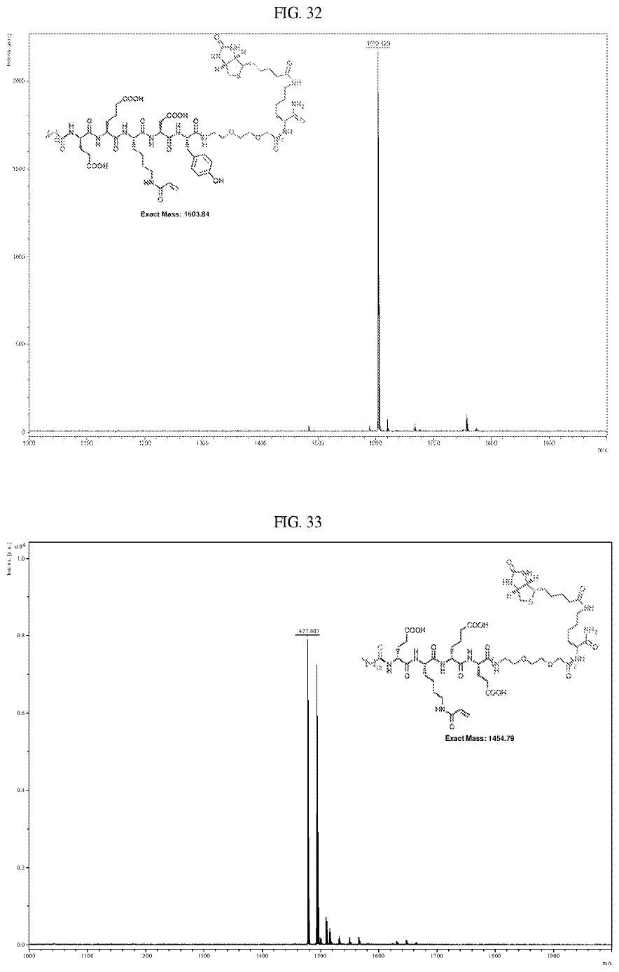

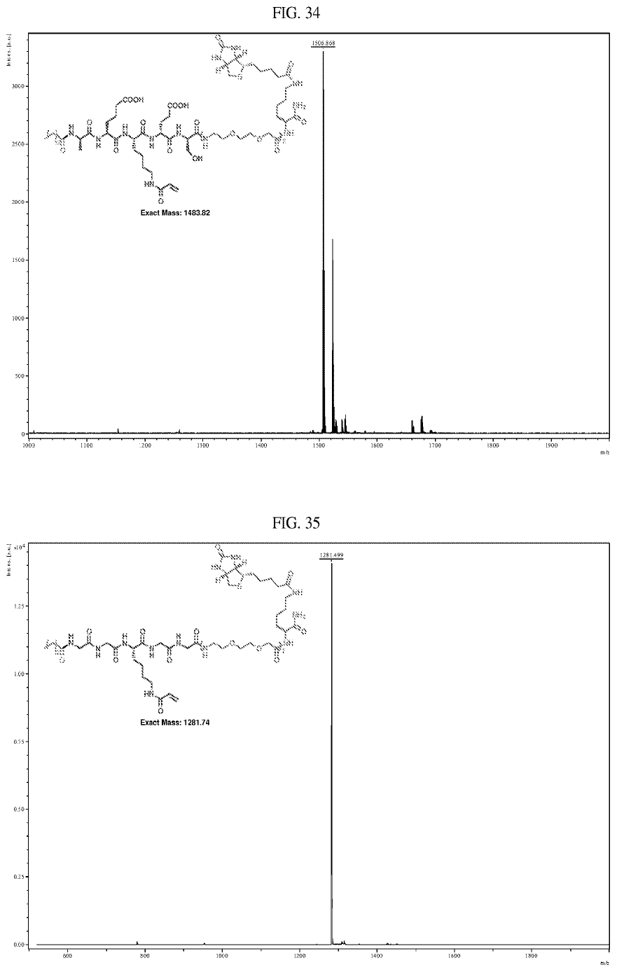

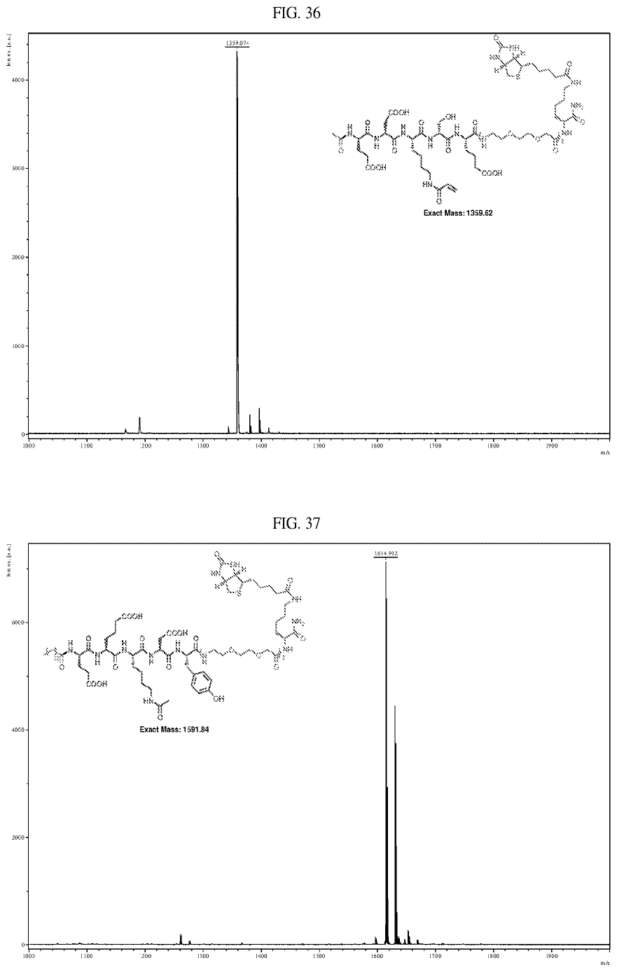

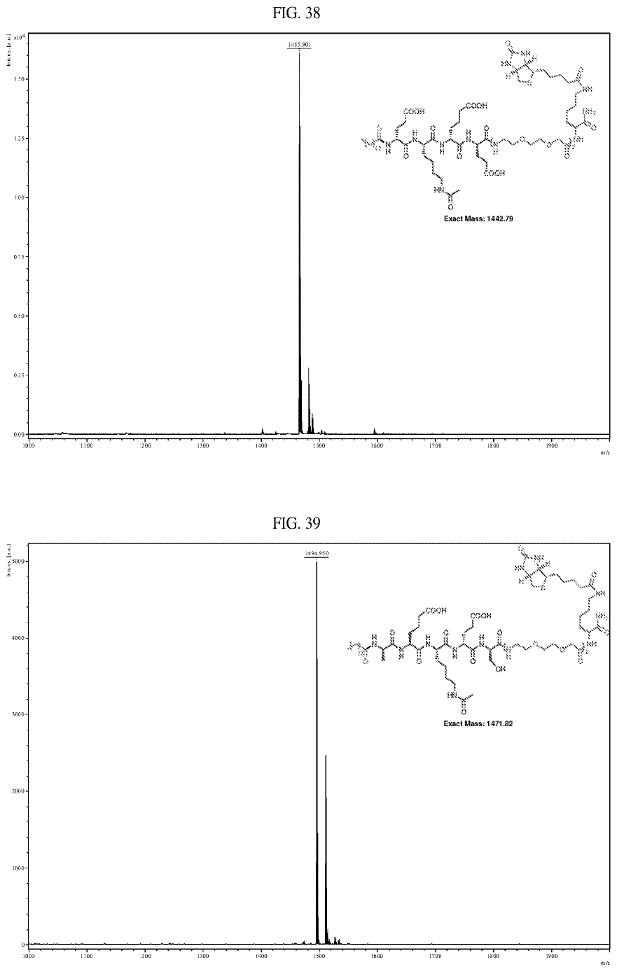

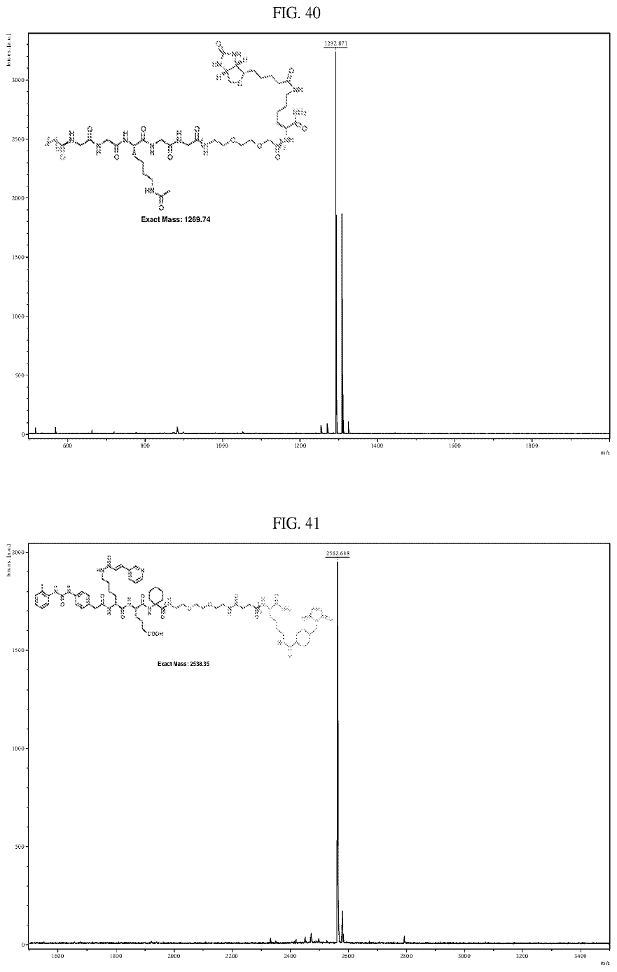

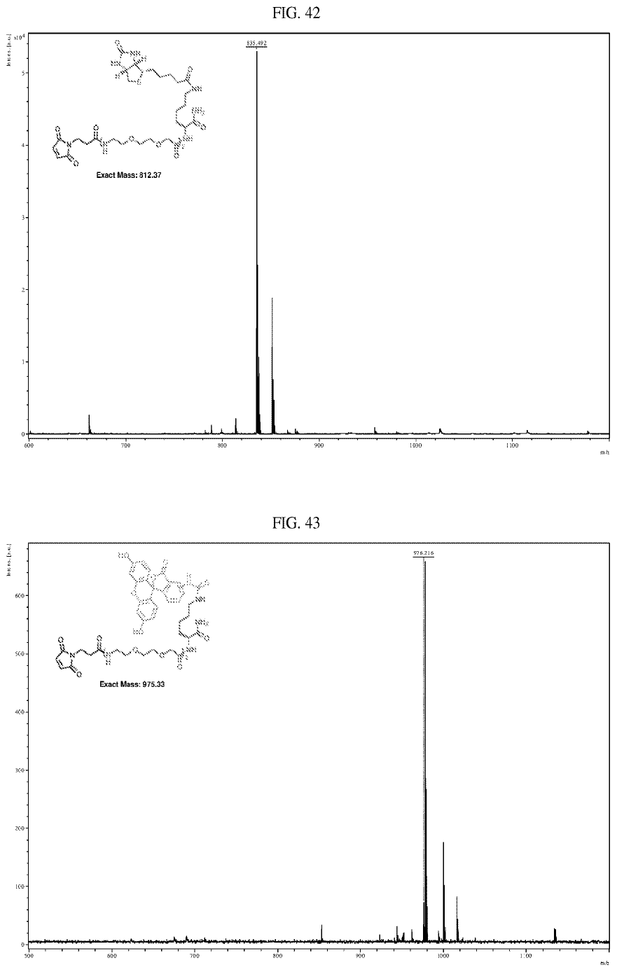

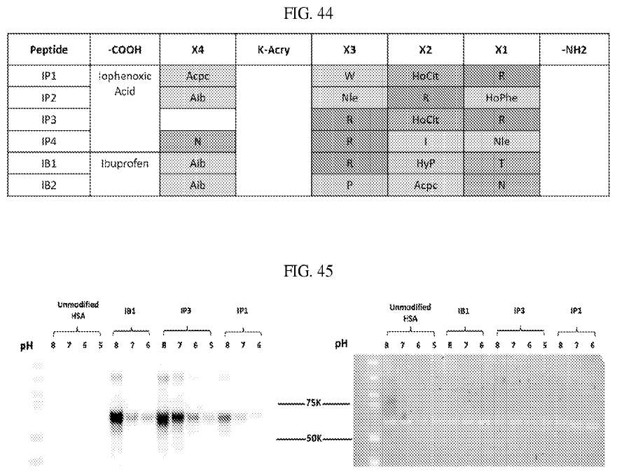

To 100 μl human serum was added 10 μl FC-LYL1 or 10 μl FC-Mal stock solution (1 mM). The albumin content in serum is presumed to be 600 μM for healthy donors. The final concentrations of albumin and FC-labeled reactive probes in the reaction mixture are calculated to be 545 M and 91 μM, respectively. The serum/ligands mixture was incubated at 37° C. in dark. The conjugation reactions were setup every 3 days, and 14 conjugation reactions were setup in total for 3 weeks. The serum mixtures were subjected to electrophoresis and Coommassie Blue Staining without further treatment. Since albumin to probe ratio was about 6:1 and there are 5 known fatty acid binding sites on each albumin molecule, fatty acid binding site:probe ratio was over 42:1. We therefore believe significant portion of the reactive probes would be sequestered in the “non-productive” (no covalent ligation) fatty acid binding sites most of the time. As a result, unlike the result shown in FIGS. 22-25 with fast ligation kinetics and good yield (in the setting of excess LYL-1 to albumin), here we observed slow reaction over days for both probes. Nonetheless, this experiment does confirm that site-specific ligation of albumin in the context of complex human serum can be achieved with LYL-1 probe, albeit taken days to complete.

### One-Pot HSA Dual-Modification by B-LYL1 and FC-Mal/B-Mal

To PBS buffer (0.1 M sodium phosphate, 0.15 M sodium chloride, pH=7.2) was added 1 μL HSA stock solution, 2 μL BLYL-1 stock solution and 5 μL FC-Mal or Mal-BT solution to prepare 200 μL conjugation solution containing 10 μM HSA. The molar ratio between HSA and B-LYL1 is 1:10, and the molar ratio between HSA and FC-Mal or B-Mal is 1:5. PBS buffer was used to replace corresponding composition for Blank and comparative experiments. The solution was incubated at room temperature for 1 hour in dark. μL conjugates were taken for electrophoresis and Western blot analysis without further purification.

### One-Pot HSA Dual-Modification by B-LYL1 and FITC

To PBS buffer (0.1 M sodium phosphate, 0.15 M sodium chloride, pH=7.2 or 8.0) was added 1 μL HSA stock solution, 2 μL 10 mM FITC stock solution and/or 2 μL 10 mM FC-LYL1 stock solution to make 200 μL conjugation solution containing 10 M HSA. The molar ration between FITC/BLYL-1 and HSA is 10:1. The solution was incubated at room temperature for 1 hour in dark. 10 μL conjugates were taken for electrophoresis and Western blot analysis without further purification.

### One-Pot HSA Dual-Modification by FC-LYL1 and Sulfo-NHS-Ester

To PBS buffer (0.1 M sodium phosphate, 0.15 M sodium chloride, pH=7.2) was added 1 μL HSA stock solution, 2 μL 10 mM FC-LYL1 DMSO stock solution. The molar ration between FITC/BLYL-1 and HSA is 10:1. Sulfo-NHS-Biotin solutions at various concentration were prepared by serial dilution using PBS buffer. The resulting solutions were added to HSA solution, which was incubated at room temperature for 1 hour in dark. 10 μL conjugates were taken for electrophoresis and Western blot analysis without further purification.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compound of Formula I: 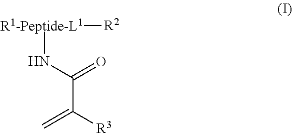
or a pharmaceutically acceptable salt thereof, wherein
R¹ is a targeting moiety;
Peptide comprises 3 to 10 amino acids;
L¹ is a linker;
R² is a drug, imaging agent, or affinity ligand; and
R³ is hydrogen or C₁₋₆ alkyl.

2. The compound of claim 1, wherein the targeting moiety is specific to a binding site of human serum albumin (HSA).

3. The compound of claim 2, wherein the HSA binding site is Sudlow I or Sudlow II.

4. The compound of any one of claims 1 to 3, wherein the targeting moiety is a fatty acid, ibuprofen, or iophenoxic acid.

5. The compound of any one of claims 1 to 3, wherein at least one amino acid of the Peptide is lysine.

6. The compound of any one of claims 1 to 5, having Formula II: 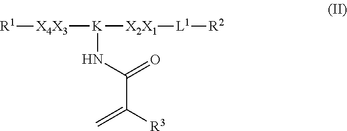
wherein
R¹ is a fatty acid;
X₄ is absent, Aad, D, d, E, e, A, a, V, v, L, I, i, L, l, M, m, F, f, Y, y, W, or w;
X₃ is Aad, D, d, E, or e;
K is lysine;
X₂ is S, s, T, t, N, n, Q, q, Aad, D, d, E, e;
X₁ is Aad, D, d, E, e, A, a, V, v, L, I, i, L, l, M, m, F, f, Y, y, W, w, S, s, T, t, N, n, Q, or q;
L¹ is a linker;
R² is the drug, imaging agent, or affinity ligand; and
R³ is hydrogen or C₁₋₆ alkyl.

7. The compound of claim 6, wherein the fatty acid is a saturated fatty acid.

8. The compound of claim 6 or 7, wherein the fatty acid is caprylic acid, capric acid, lauric acid, myristic acid, palmitic acid, stearic acid, arachidic acid, behenic acid, lignoceric acid, or cerotic acid.

9. The compound of any one of claims 6 to 8, wherein the fatty acid is myristic acid.

10. The compound of any one of claims 6 to 9, wherein at least two of X₄, X₃, X₂ and X₁ are each independently Aad, D, d, E, or e.

11. The compound of any one of claims 6 to 10, wherein
X₄ is absent, e or a;
X₃ is Aad, D, or e;
X₂ is s, Aad, D, or e; and
X₁ is Aad, Y, e, or s,
wherein at least two of X₄, X₃, X₂ and X₁ are each independently Aad, D, or e.

12. The compound of any one of claims 1 to 11, wherein R³ is hydrogen or methyl.

13. The compound of any one of claims 1 to 12, wherein R³ is hydrogen.

14. The compound of any one of claims 6 to 13, having Formula IIa: 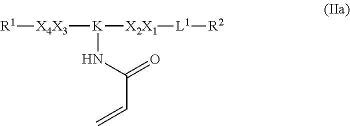
wherein
R¹ is myristic acid;
X₄ is absent, e or a;
X₃ is Aad, D, or e;
K is lysine;
X₂ is s, Aad, D, or e;
X₁ is Aad, Y, e, or s;
L¹ is a linker; and
R² is the drug, imaging agent, or affinity ligand,
wherein at least two of X₄, X₃, X₂ and X₁ are each independently Aad, D, or e.

15. The composition of any one of claims 1 to 14, wherein the compound is: 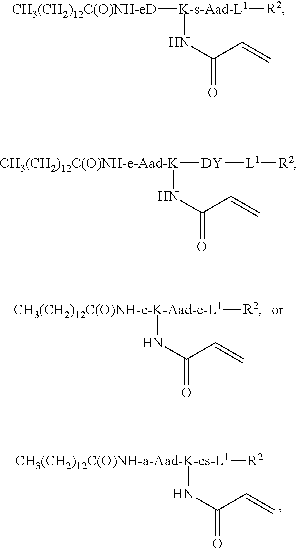
wherein
L¹ is the linker 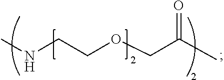
and
R² is the drug, imaging agent, or affinity ligand.

16. The compound of any one of claims 6 to 13, having Formula III: 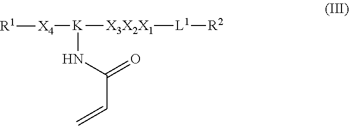
wherein
R¹ is ibuprofen or iophenoxic acid;
X₄ is absent, Acpc, Aib or N;
K is lysine;
X₃ is W, Nle, R, or P;
X₂ is HoCit, R, I, HyP, or Acpc;
X₁ is R, HoPhe, Nle, T, or N;
L¹ is a linker; and
R² is the drug, imaging agent, or affinity ligand,
wherein at least one of X₄, X₃, X₂ and X₁ is Acpc, W, Aib, Nle, HoPhe, or I.

17. The compound of claim 16, having Formula III: 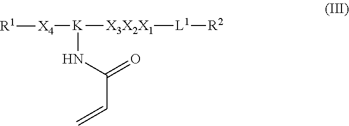
wherein
R¹ is ibuprofen;
X₄ is absent, Acpc, Aib or N;
K is lysine;
X₃ is W, Nle, or R;
X₂ is HoCit, R, or I;
X₁ is R, HoPhe, or Nle;
L¹ is a linker; and
R² is a drug, imaging agent, or affinity ligand,
wherein at least two of X₄, X₃, X₂ and X₁ are each independently Acpc, W, Aib, Nle, HoPhe, or I.

18. The composition of claim 16 or 17, wherein the compound is: 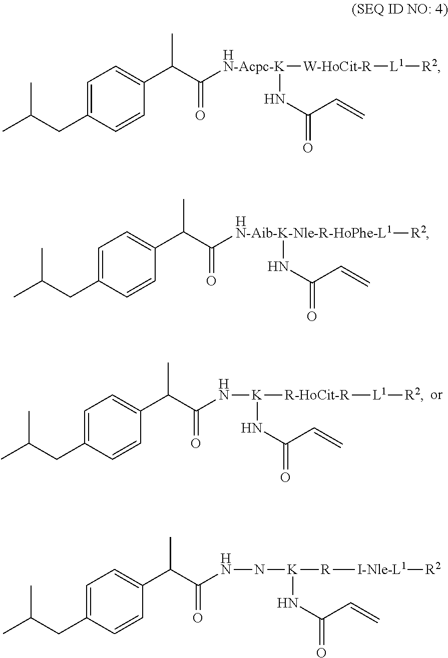
wherein
L¹ is the linker 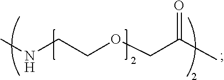
and
R² is the drug, imaging agent, or affinity ligand.

19. The compound of claim 16, having Formula III: 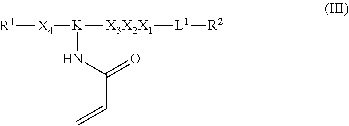
wherein
R¹ is iophenoxic acid;
X₄ is Aib;
K is lysine;
X₃ is R, or P;
X₂ is HyP, or Acpc;
X₁ is T, or N;
L¹ is a linker; and
R² is a drug, imaging agent, or affinity ligand,
wherein at least one of X₄, X₃, X₂ and X₁ is Acpc, or Aib.

20. The composition of claim 16 or 19, wherein the compound is: 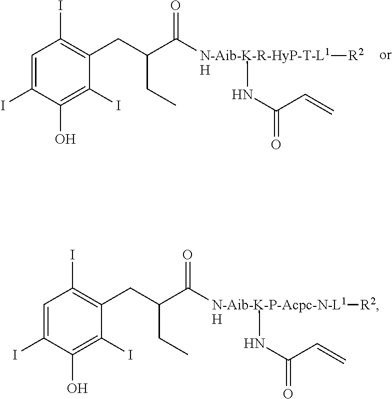
wherein
L¹ is the linker 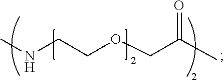
and
R² is the drug, imaging agent, or affinity ligand.

21. A pharmaceutical composition comprising a compound of any one of claims 1 to 20, and a pharmaceutically acceptable excipient.

22. A conjugate of Formula IV: 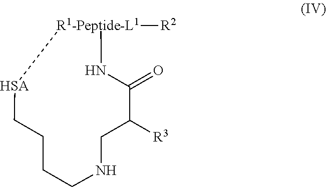
wherein
R¹ is a targeting moiety;
Peptide comprises 3 to 10 amino acids;
L¹ is a linker;
R² is a drug, imaging agent, or affinity ligand;
R³ is hydrogen or C₁₋₆ alkyl; and
HSA is human serum albumin,
wherein the targeting moiety is bound to a first binding site of the HSA.

23. The conjugate of claim 22, having Formula IVa: 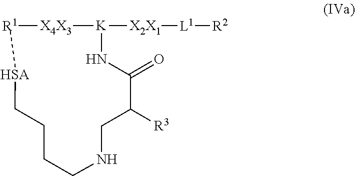
wherein
R¹ is a fatty acid;
X₄ is absent, Aad, D, d, E, e, A, a, V, v, L, I, i, L, l, M, m, F, f, Y, y, W, or w;
X₃ is Aad, D, d, E, or e;
K is lysine;
X₂ is S, s, T, t, N, n, Q, q, Aad, D, d, E, e;
X₁ is Aad, D, d, E, e, A, a, V, v, L, I, i, L, l, M, m, F, f, Y, y, W, w, S, s, T, t, N, n, Q, or q;
L¹ is a linker;
R² is a drug, imaging agent, or affinity ligand;
R³ is hydrogen or C₁₋₆ alkyl; and
HSA is human serum albumin,
wherein the fatty acid is non-covalently bound to the first binding site of the HSA.

24. The conjugate of claim 22 or 23, having Formula IVb: 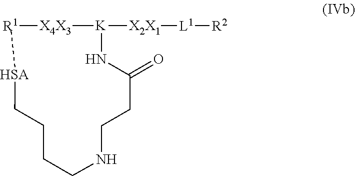
wherein
R¹ is myristic acid;
X₄ is absent, e or a;
X₃ is Aad, D, or e;
K is lysine;
X₂ is s, Aad, D, or e;
X₁ is Aad, Y, e, or s;
L¹ is the linker 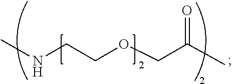
R² is the drug, imaging agent, or affinity ligand; and
HSA is human serum albumin,
wherein the myristic acid is non-covalently bound to the first binding site of the HSA, and
wherein at least two of X₄, X₃, X₂ and X₁ are each independently Aad, D, or e.

25. The conjugate of claim 22, having Formula IVc: 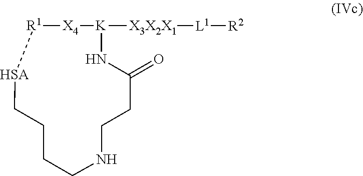
wherein
R¹ is ibuprofen;
X₄ is absent, Acpc, Aib or N;
K is lysine;
X₃ is W, Nle, or R;
X₂ is HoCit, R, or I;
X₁ is R, HoPhe, or Nle;
L¹ is the linker 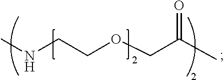
R² is the drug, imaging agent, or affinity ligand; and
HSA is human serum albumin,
wherein the ibuprofen is non-covalently bound to the first binding site of the HSA, and
wherein at least two of X₄, X₃, X₂ and X₁ are each independently Acpc, W, Aib, Nle, HoPhe, or I.

26. The conjugate of claim 22, having Formula IVc: 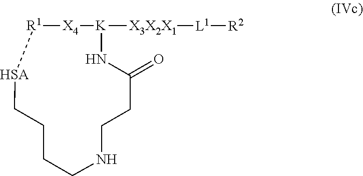
wherein
R¹ is iophenoxic acid;
X₄ is Aib;
K is lysine;
X₃ is R, or P;
X₂ is HyP, or Acpc;
X₁ is T, or N;
L¹ is the linker 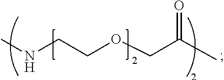
R² is the drug, imaging agent, or affinity ligand; and
HSA is human serum albumin,
wherein the iophenoxic acid is non-covalently bound to the first binding site of the HSA, and
wherein at least one of X₄, X₃, X₂ and X₁ is Acpc, or Aib.

27. A method of preparing a conjugate of any one of claims 22 to 26, comprising:
forming a reaction mixture comprising a compound of any one of claims 1 to 20, and human serum albumin, wherein the reaction mixture is at a pH of from 6 to 9, thereby forming the conjugate of any one of claims 22 to 26.

28. The method of claim 27, wherein the reaction mixture is at a pH of from 6.5 to 7.5.

29. The method of claim 27 or 28, wherein the reaction mixture is at a pH of about 7.2.

30. The method of claim 27 or 28, comprising forming the reaction mixture comprising the compound of claim 14, and human serum albumin, wherein the reaction mixture is at a pH of about 7.2, thereby forming the conjugate of claim 24.

31. A method of treating a disease or condition, comprising administering to a subject in need thereof, a therapeutically effective amount of a compound of any one of claims 1 to 20, forming the conjugate of any one of claims 22 to 26, thereby treating the disease or condition.


SEQUENCE LISTING (5 sequences)

SEQ ID NO: 1 - protein, 600 aa, synthetic construct
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLV

SEQ ID NO: 2 - protein, 11 aa, synthetic construct
FPKAEFAEVSK

SEQ ID NO: 3 - protein, 15 aa, synthetic construct
KVPQVSTPTLVEVSR

SEQ ID NO: 4 - protein, 4 aa, synthetic construct
NKRI

SEQ ID NO: 5 - protein, 5 aa, synthetic construct
  SITE 5: note = X represents Aad (2-Aminoadipic acid)
EDKSX